(19) United States
(12) Patent Application Publication
(10) Pub. No.: US 2025/0161338 A1
(43) Pub. Date: May 22, 2025

(54) Compounds for Anti-aging Intervention

(71) Applicant: AGENCY FOR SCIENCE, TECHNOLOGY AND RESEARCH, Singapore (SG)

(72) Inventors: Mohammad Alfatah, Singapore (SG); Frank Eisenhaber, Singapore (SG); Yizhong Zhang, Singapore (SG)

(21) Appl. No.: 18/839,102

(22) PCT Filed: Feb. 17, 2023
(86) PCT No.: PCT/SG2023/050096

§ 371 Date: Aug. 16, 2024

(30) Foreign Application Priority Data
Feb. 17, 2022 (SG) 10202201536U

(51) Int. Cl. A61K 31/7004; A61K 31/40; A61K 31/513; A61K 31/519; A61K 31/5377; A61K 31/555; A61K 31/7024; A61K 31/7034; A61P 39/00

(52) U.S. Cl. CPC A61K 31/7004; A61K 31/40; A61K 31/513; A61K 31/519; A61K 31/5377; A61K 31/555; A61K 31/7024; A61K 31/7034; A61P 39/00

(57) ABSTRACT

The present invention relates to the use of a compound of formula (I):
to extend the chronological lifespan of a cell, a method for extending the chronological lifespan of a cell, the compound for use in extending the chronological lifespan of a cell and the use of the compound in the manufacture of a medicament for extending the chronological lifespan of a cell.

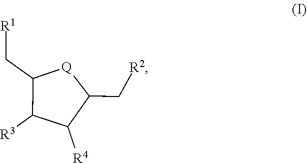

### TECHNICAL FIELD

The present invention relates to the use of a compound to extend the chronological lifespan of a cell, a method for extending the chronological lifespan of a cell, a compound for use in extending the chronological lifespan of a cell and the use of a compound in the manufacture of a medicament for extending the chronological lifespan of a cell.

### BACKGROUND

The aging population continues to grow at an unprecedented rate worldwide. The growing fraction of the elderly (greater than 65 years in age) among the total population, from about 10% worldwide (above 20% in first world countries) in 2020 predicted to be 16% in 2050, as well as their increasing life expectancy (by about 10 years since 1960 in first world countries), confronts the world with increasingly difficult problems. The increase in the number of years lived is not a representation of the health status of the elderly. There is a remarkable gap of almost ten years between the healthspan, which is the period essentially free from diseases, and the actual lifespan. Aging is associated with a decline in cellular functions, damage accumulation, and an increasing probability of chronic diseases that lead to system collapse and eventual death. The prevalence of age-related pathologies later in life has a significant impact on life quality and healthcare costs. Elders tend to have costly chronic diseases such as cancer, neurodegenerative and cardiovascular diseases and other metabolic syndromes, which severely hamper their quality of life and ability to work. As such, this puts tremendous pressure on the healthcare system and the working population. Aging has become a global challenge in the 21^{st} century.

The current medical approaches to prevent age-related pathologies are recommendations for a healthy lifestyle, including exercise and diet. However, these interventions alone are not sufficient to prevent the onset of age-related diseases. Thus, there is an urgent need for interventions with an effective anti-aging compound that can be exploited as a geroprotector to delay aging and prolong health span.

Increasing efforts are directed to understand cellular aging processes affecting the highly interconnected and functionally redundant gene and protein interactions network. Despite the complexity of aging, recent research in different model systems, including mammals, has demonstrated that delayed aging and increased healthspan are feasible by anti-aging interventions such as rapamycin drug administration application and calorie (glucose) restriction. Expanding the repertoire of anti-aging compounds that can be utilized as geroprotecting therapeutics and reducing their unwanted side effects is one of the promising strategies that can delay aging and prolong health span. However, identification of such compounds has not been easy due to limited ways in which anti-aging compounds can be identified.

In view of the above, there is a need to find new compounds that are capable of extending cell life span that overcomes or at least ameliorate, one or more of the limitations described above.

### SUMMARY

In an aspect, there is provided the use of a compound selected from the group consisting of a compound having the following formula (I), plicamycin, copanlisib dihydrochloride, nedaplatin, hemin, 5-fluorouracil, atorvastatin, methotrexate, and any mixture thereof, to extend the chronological lifespan of a cell:

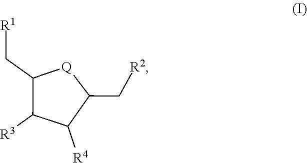

wherein in formula (I),
R¹, R², R³ and R⁴ is each independently -QH, —NR⁵R⁶, or —OC(O)R⁵;
R⁵ and R⁶ is each independent —H or an optionally substituted alkyl; and
Q is O or S.

In another aspect, there is provided a method of extending the chronological lifespan of a cell, comprising the step of contacting a cell with a compound selected from the group consisting of a compound having the following formula (I), plicamycin, copanlisib dihydrochloride, nedaplatin, hemin, 5-fluorouracil, atorvastatin, methotrexate, and any mixture thereof:

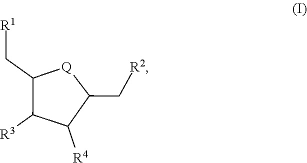

wherein in formula (I),
R¹, R², R³ and R⁴ is each independently -QH, —NR⁵R⁶, or —OC(O)R⁵;
R⁵ and R⁶ is each independent —H or an optionally substituted alkyl; and
Q is O or S.

In another aspect, there is provided a compound selected from the group consisting of a compound having the following formula (I), plicamycin, copanlisib dihydrochloride, nedaplatin, hemin, 5-fluorouracil, atorvastatin, methotrexate, and any mixture thereof for use in extending the chronological lifespan of a cell:

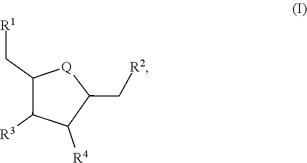

wherein in formula (I),
R¹, R², R³ and R⁴ is each independently -QH, —NR⁵R⁶, or —OC(O)R⁵;
R⁵ and R⁶ is each independent —H or an optionally substituted alkyl; and
Q is O or S.

In another aspect, there is provided the use of a compound selected from the group consisting of a compound having the following formula (I), plicamycin, copanlisib dihydrochloride, nedaplatin, hemin, 5-fluorouracil, atorvastatin, methotrexate, and any mixture thereof in the manufacture of a medicament for extending the chronological lifespan of a cell:

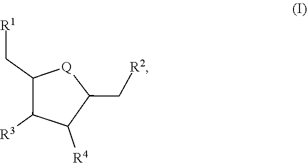

wherein in formula (I),
R¹, R², R³ and R⁴ is each independently -QH, —NR⁵R⁶, or —OC(O)R⁵;
R⁵ and R⁶ is each independent —H or an optionally substituted alkyl; and
Q is O or S.

Advantageously, the compound as defined above may increase the survival rate of a cell. Further advantageously, the compound as defined above may extend the lifespan of a cell even at a late stage of chronological aging. Advantageously, the compound as defined above may increase the survival rate of a cell by a mechanism that may be different to conventionally known anti-aging agents, thereby providing an effective alternative to conventionally known anti-aging agents.

### Definitions

“Alkyl” as a group or part of a group refers to a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit the inclusion of additional, unrecited elements.

As used herein, the term “about”, in the context of concentrations of components of the formulations, typically means ±5% of the stated value, more typically ±4% of the stated value, more typically ±3% of the stated value, more typically, ±2% of the stated value, even more typically ±1% of the stated value, and even more typically ±0.5% of the stated value.

Throughout this disclosure, certain embodiments may be disclosed in range formats. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether the excised material is specifically recited herein.

### Detailed Disclosure of Optional Embodiments

There is provided the use of a compound selected from the group consisting of a compound having the following formula (I), plicamycin, copanlisib dihydrochloride, nedaplatin, hemin, 5-fluorouracil, atorvastatin, methotrexate, and any mixture thereof, to extend the chronological lifespan of a cell:

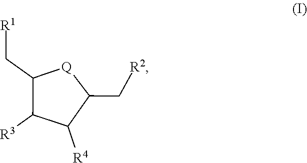

wherein in formula (I),
R¹, R², R³ and R⁴ may independently be -QH, —NR⁵R⁶, or —OC(O)R⁵;
R⁵ and R⁶ may independent be —H or an optionally substituted alkyl; and
Q may be O or S.

There is also provided a method of extending the chronological lifespan of a cell, comprising the step of contacting a cell with a compound selected from the group consisting of a compound having the following formula (I), plicamycin, copanlisib dihydrochloride, nedaplatin, hemin, 5-fluorouracil, atorvastatin, methotrexate, and any mixture thereof:

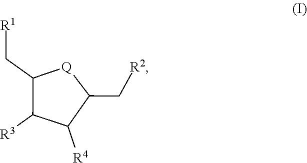

wherein in formula (I),
R¹, R², R³ and R⁴ may independently be -QH, —NR⁵R⁶, or —OC(O)R⁵;
R⁵ and R⁶ may independent be —H or an optionally substituted alkyl; and
Q may be O or S.

There is also provided a compound selected from the group consisting of a compound having the following formula (I), plicamycin, copanlisib dihydrochloride, nedaplatin, hemin, 5-fluorouracil, atorvastatin, methotrexate, and any mixture thereof for use in extending the chronological lifespan of a cell:

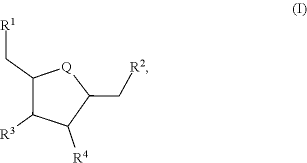

wherein in formula (I),
R¹, R², R³ and R⁴ may independently be -QH, —NR⁵R⁶, or —OC(O)R⁵;
R⁵ and R⁶ may independent be —H or an optionally substituted alkyl; and
Q may be O or S.

There is also provided the use of a compound selected from the group consisting of a compound having the following formula (I), plicamycin, copanlisib dihydrochloride, nedaplatin, hemin, 5-fluorouracil, atorvastatin, methotrexate, and any mixture thereof in the manufacture of a medicament for extending the chronological lifespan of a cell:

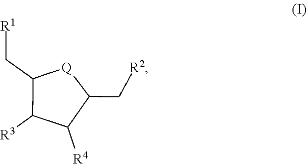

wherein in formula (I),
R¹, R², R³ and R⁴ may independently be -QH, —NR⁵R⁶, or —OC(O)R⁵;
R⁵ and R⁶ may independent be —H or an optionally substituted alkyl; and
Q may be O or S.

Advantageously, the compound selected from the group consisting of a compound of formula (I), plicamycin, copanlisib dihydrochloride, nedaplatin, hemin, 5-fluorouracil, atorvastatin, methotrexate, and any mixture thereof, may increase the survival rate of a cell.

Advantageously, the survival of cells supplemented with the compound of formula (I) (4 mM and 8 mM) may be approximately 2.5-fold higher compared to non-supplemented cells on day 4, the survival of cells supplemented with the compound of formula (I) (4 mM and 8 mM) may be approximately 4-fold higher compared to non-supplemented cells on day 7, the survival of cells supplemented with the compound of formula (I) (4 mM) may be approximately 6-fold higher compared to non-supplemented cells on day 14, the survival of cells supplemented with the compound of formula (I) (8 mM) may be approximately 8-fold higher compared to non-supplemented cells on day 14, the survival of cells supplemented with the compound of formula (I) (4 mM) may be approximately 7-fold higher compared to non-supplemented cells on day 21, or the survival of cells supplemented with the compound of formula (I) (8 mM) may be approximately 10-fold higher compared to non-supplemented cells on day 21.

Advantageously, plicamycin, copanlisib dihydrochloride, nedaplatin, hemin, 5-fluorouracil, atorvastatin, and methotrexate may increase cellular lifespan under these conditions, similarly to the compound of formula (I).

The use, method or compound as defined above may comprise contacting the cell with the compound selected from the group consisting of a compound of formula (I), plicamycin, copanlisib dihydrochloride, nedaplatin, hemin, 5-fluorouracil, atorvastatin, methotrexate, and any mixture thereof.

The use, method or compound as defined above may comprise contacting the cell with the compound selected from the group consisting of a compound of formula (I), plicamycin, copanlisib dihydrochloride, nedaplatin, hemin, 5-fluorouracil, atorvastatin, methotrexate, and any mixture thereof in vitro, in vivo or ex vivo.

In formula (I), each carbon atom labelled with an asterisk as indicated below, may be a stereocenter. Each stereocenter may independently be in the (R)- or (S)-orientation.

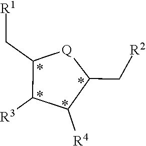

The compound of formula (I) may have the following formula (Ia), (Ib) or (Ic):

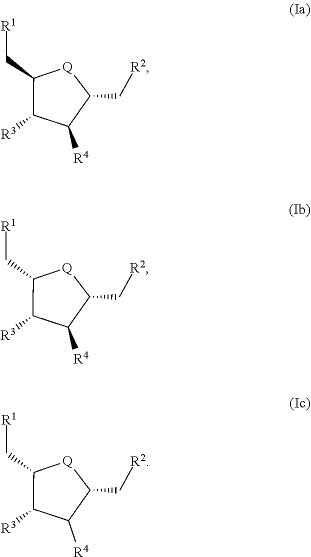

The optionally substituted alkyl maybe a methyl, ethyl, n-propyl, isopropyl, n-butyl, sec-butyl, isobutyl or tert-butyl group. The alkyl may be optionally substituted with a halogen, hydroxy or amine.
R¹, R², R³ and R⁴ may independently be —OH, —O—C(O)—CH₃ or —NH₂.
R¹, R², R³ and R⁴ may all be —OH.
R¹, R², R³ and R⁴ may all be —OC(O)R⁵.
R¹ may be NH₂ and R², R³ and R⁴ may all be —OH.
Q may be O.

The compound of formula (I) may be selected from the group consisting of 2,5-anhydro-D-mannitol, 2,5-anhydro-D-mannitol tetraacetate, 2,5-anhydro-D-glucitol, and 1-amino-2,5-anhydro-D-glucitol.

The compound of formula (I) may be selected from the group consisting of:

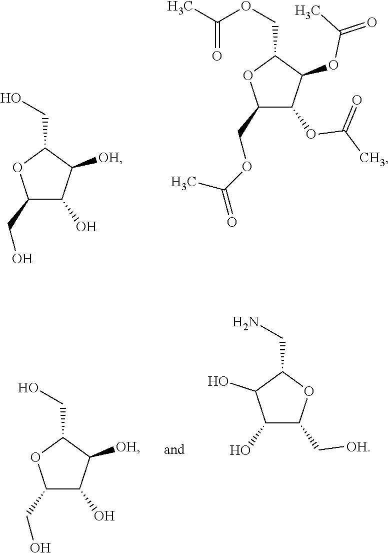 [SMILES: OC[C@H]1O[C@H](CO)[C@@H](O)[C@@H]1O ; CC(=O)OC[C@H]1O[C@H](COC(C)=O)[C@@H](OC(C)=O)[C@@H]1OC(C)=O ; OC[C@@H]1O[C@H](CO)[C@@H](O)[C@@H]1O ; NC[C@@H]1O[C@H](CO)[C@H](O)C1O]

Plicamycin may have the following structure:

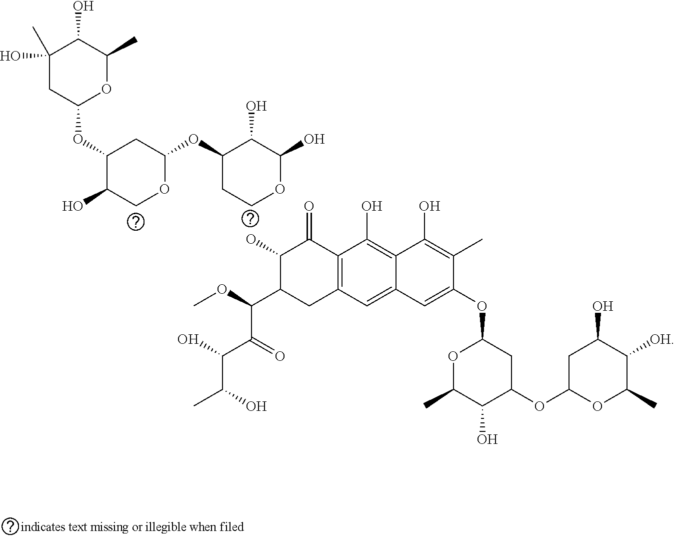

Copanlisib dihydrochloride may have the following structure:

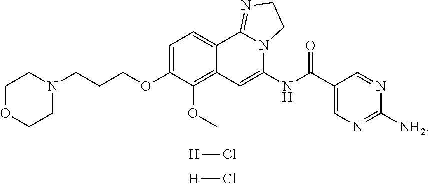

Nedaplatin may have the following structure:

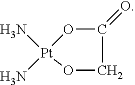

Hemin may have the following structure:

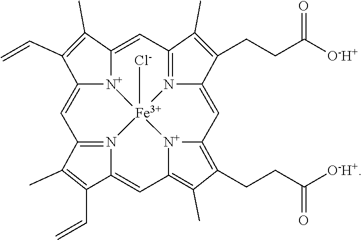

5-fluorouracil may have the following structure:

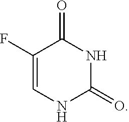

Atorvastatin may have the following structure:

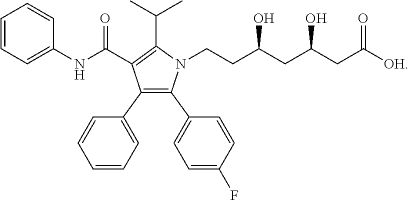 [SMILES: CC(C)c1c(C(=O)Nc2ccccc2)c(-c2ccccc2)c(-c2ccc(F)cc2)n1CC[C@@H](O)C[C@@H](O)CC(=O)O]

Methotrexate may have the following structure:

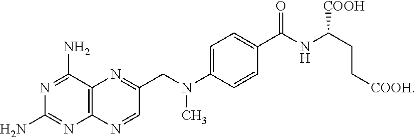 [SMILES: CN(Cc1cnc2nc(N)nc(N)c2n1)c1ccc(C(=O)N[C@@H](CCC(=O)O)C(=O)O)cc1]

The use, method or compound as defined above may comprise contacting the cell with one or more of the individual compounds falling within the scope of the compound selected from the group consisting of a compound of formula (I), plicamycin, copanlisib dihydrochloride, nedaplatin, hemin, 5-fluorouracil, atorvastatin, methotrexate, or any mixture thereof.

The compound selected from the group consisting of a compound of formula (I), plicamycin, copanlisib dihydrochloride, nedaplatin, hemin, 5-fluorouracil, atorvastatin, methotrexate, and any mixture thereof, may be contacted with the cell at various concentrations. The compound selected from the group consisting of a compound of formula (I), plicamycin, copanlisib dihydrochloride, nedaplatin, hemin, 5-fluorouracil, atorvastatin, methotrexate, and any mixture thereof, may be contacted with the cell at concentrations in the range of about 0 nM to about 2M, more than 0 nM to about 2 M or about 0.0001 nM to about 2 nM. The cells may be treated with the lifespan modulating agent at a concentration in the range of 0 nM to about 2M, 0 nM to about 0.0001 nM, 0 nM to about 0.001 nM, 0 nM to about 0.01 nM, 0 nM to about 0.1 nM, 0 nM to about 1 nM, 0 nM to about 10 nM, 0 nM to about 100 nM, 0 nM to about 1 μM, 0 nM to about 10 μM, 0 nM to about 100 μM, 0 nM to about 1 mM, 0 nM to about 10 mM, 0 nM to about 100 mM, 0 nM to about 1M, about 0.0001 nM to about 0.001 nM, about 0.0001 nM to about 0.01 nM, about 0.0001 nM to about 0.1 nM, about 0.0001 nM to about 1 nM, about 0.0001 nM to about 10 nM, about 0.0001 nM to about 100 nM, about 0.0001 nM to about 1 μM, about 0.0001 nM to about 10 μM, about 0.0001 nM to about 100 μM, about 0.0001 nM to about 1 mM, about 0.0001 nM to about 10 mM, about 0.0001 nM to about 100 mM, about 0.0001 nM to about 1M, about 0.0001 nM to about 2 M, about 1 μM to about 10 μM, about 1 μM to about 100 μM, about 1 μM to about 1 mM, about 1 μM to about 10 mM, about 1 μM to about 100 mM, about 1 μM to about 1M, about 1 μM to about 2 M, about 10 μM to about 100 μM, about 10 μM to about 1 mM, about 10 μM to about 10 mM, about 10 μM to about 100 mM, about 10 μM to about 1M, about 10 μM to about 2 M, about 100 μM to about 1 mM, about 100 μM to about 10 mM, about 100 μM to about 100 mM, about 100 μM to about 1M, about 100 μM to about 2 M, about 1 mM to about 10 mM, about 1 mM to about 100 mM, about 1 mM to about 1M, about 1 mM to about 2 M, about 10 mM to about 100 mM, about 10 mM to about 1M, about 10 mM to about 2 M, about 100 mM to about 1M, about 100 mM to about 2 M, or about 1 M to about 2 M.

The compound selected from the group consisting of a compound of formula (I), plicamycin, copanlisib dihydrochloride, nedaplatin, hemin, 5-fluorouracil, atorvastatin, methotrexate, and any mixture thereof, may be contacted with the cell for various durations. The compound selected from the group consisting of a compound of formula (I), plicamycin, copanlisib dihydrochloride, nedaplatin, hemin, 5-fluorouracil, atorvastatin, methotrexate, and any mixture thereof, may be contacted with the cell for a duration in the range of about 1 day to about 30 days, about 1 day to about 7 days, about 1 day to about 14 days, about 1 day to about 21 days, about 1 day to about 30 days, about 7 days to about 14 days, about 7 days to about 21 days, about 7 days to about 30 days, about 14 days to about 21 days, about 14 days to about 30 days, or about 21 days to about 30 days. The compound selected from the group consisting of a compound of formula (I), plicamycin, copanlisib dihydrochloride, nedaplatin, hemin, 5-fluorouracil, atorvastatin, methotrexate, and any mixture thereof, may be contacted with the cell for a duration of more than 1 day, more than 7 days, more than 14 days, more than 21 days, or more than 30 days.

The cell may be a fungal cell, bacterial cell or animal cell. The fungal cell may be a yeast cell. The yeast cell may be a cell of yeast selected from the group consisting of Saccharomyces cerevisiae, Saccharomyces pombe, Pichia pastoris and any mixture thereof.

The animal cells may be a mammalian cell. The mammalian cell may be a human cell. The human cell may be a primary cell or an immortal cell. The human cell may be a cancer cell. The human cell may be selected from the group consisting of a kidney cell, liver cell, muscle cell, lung cell, neuron, retinal cell, skin cell and any mixture thereof. The cells may be human embryonic kidney 293 (HEK293) cells or lung epithelial (A549 cells) or fibroblast (IMR90) cells.

Advantageously, the compound selected from the group consisting of a compound of formula (I), plicamycin, copanlisib dihydrochloride, nedaplatin, hemin, 5-fluorouracil, atorvastatin, methotrexate, and any mixture thereof, may be used in fungal cells, bacterial cells and/or animal cells. Although there are critical differences in aging of yeast and animals, the compound selected from the group consisting of a compound of formula (I), plicamycin, copanlisib dihydrochloride, nedaplatin, hemin, 5-fluorouracil, atorvastatin, methotrexate, and any mixture thereof, may advantageously extend the chronological lifespan of both yeast and animals, and in different cell types within each category.

The extension of the chronological lifespan of a cell may result in treatment of an aging-associated condition in a subject in need thereof.

The aging-associated condition may be diabetic complications, retinopathy, atherosclerosis, hypertension, obesity, cancer, benign prostate hyperplasia, Alzheimer and Parkinson diseases, age-related macular degeneration, osteoarthritis, osteoporosis, sarcopenia and seborrheic keratosis

Advantageously, the compound selected from the group consisting of a compound of formula (I), plicamycin, copanlisib dihydrochloride, nedaplatin, hemin, 5-fluorouracil, atorvastatin, methotrexate, and any mixture thereof, may delay the aging-process and prevent age-related diseases. Further advantageously, the compound selected from the group consisting of a compound of formula (I), plicamycin, copanlisib dihydrochloride, nedaplatin, hemin, 5-fluorouracil, atorvastatin, methotrexate, and any mixture thereof, may prolong health.

### BRIEF DESCRIPTION OF DRAWINGS

The accompanying drawings illustrate a disclosed embodiment and explain the principles of the disclosed embodiment. It is to be understood, however, that the drawings are designed for purposes of illustration only, and not as a definition of the limits of the invention.

FIG. 1 refers to a flowchart of the propidium iodide fluorescence-based method and traditional outgrowth methods (outgrowth in YPD liquid medium and outgrowth in YPD agar medium/spotting assay) for screening chemical agents to identify anti-aging compounds. (102) refers to yeast, (104) refers to yeast cells grow onto YPD agar medium at 30° C. for 2 days, (106) refers to yeast cells inoculation, (108) refers to yeast culture, (110) refers to yeast cells grow in SD medium at 30° C. for 12 to 16 hours, (112) refers to 0.2 OD600 nm yeast culture, (114) refers to chemical agents in 96-well plate, (116) refers to yeast cells incubate with chemicals and grow in SD medium (200 μL), (118) refers to propidium iodide (PI) fluorescence method, (120) refers to an aliquot of cells stained with PI (5 μg/mL) in a fresh 96-well plate at 30° C., (122) refers to 15 minutes, (124) refers to PI fluorescence reading (Excitation 535 nm/Emission 617 nm) by a microplate reader, (126) refers to outgrowth in liquid medium, (128) refers to an aliquot of cells transfer into YPD medium (200 μL) in a fresh 96-well plate and incubate at 30° C., (130) refers to 24 hours, (132) refers to cell outgrowth reading (OD600 nm) by a microplate reader, (134) refers to outgrowth onto agar medium, (136) refers to an aliquot of cells spotted onto YPD agar medium and incubated at 30° C., (138) refers to 48 hours, and (140) refers to cell outgrowth visualize by agar plate imaging.

FIG. 2 refers to the high-throughput screening (HTS) outcome of various chemicals to identify novel anti-aging compounds. [FIG. 2A] refers to a graph evaluating the CLS of different chemical agents using the PI fluorescence-based method. Cell survival was quantified at chronological age point Day 7, and the growth time point 72 hours was considered as Day 1. (202) refers to rifamycin (sodium), (204) refers to DL-serine, (206) refers to β-estradiol 17-acetate, (208) refers to rapamycin, (210) refers to melanin, (212) refers to acivicin, (214) refers to 2,5-anhydro-mannitol, (216) refers to epiberberine (chloride), (218) refers to engeletin, (220) refers to selenomethionine, and (222) refers to L-selenomethionine. [FIG. 2B] refers to a graph evaluating the CLS of aged cells using the outgrowth method in YPD liquid medium. The growth time point 72 hours was considered as Day 1. (202) refers to rifamycin (sodium), (204) refers to DL-serine, (206) refers to β-estradiol 17-acetate, (208) refers to rapamycin, (210) refers to melanin, (212) refers to acivicin, (214) refers to 2,5-anhydro-mannitol, (216) refers to epiberberine (chloride), (218) refers to engeletin, (220) refers to selenomethionine, and (222) refers to L-selenomethionine.

FIG. 3 refers to a schematic representation for determining anti-aging compounds that extend the CLS of yeast. (302) refers to live cell, (304) refers to growth phase, (306) refers to nutrient depletion, (308) refers to stationary phase, (310) refers to chronological lifespan, (312) refers to cell death, (314) refers to anti-aging compound, and (316) refers to extension of chronological lifespan.

FIG. 4 refers to a graph evaluating the effect of different concentrations of 2,5-AM on cell growth at different time points (24 hours, 48 hours, and 72 hours). All data are represented as mean±SD. N.s. denotes non-significance.

FIG. 5 refers to a graph evaluating the effect of different concentrations of 2,5-AM on CLS at different chronological age points. The growth time point 72 hours was considered as Day 1. All data are represented as mean±SD. **P<0.01 and ****P<0.0001 are based on two-way ANOVA, followed by Dunnett's multiple comparisons test. N.s. denotes non-significance.

FIG. 6 shows a photograph of outgrowth in 96-well plates at Day 1, 4, 7 and 10 with different concentrations of 2,5-AM at various chronological age points after incubation for 24 hours at 30° C.

FIG. 7 refers to a graph evaluating the effects of different concentrations of 2,5-AM on the outgrowth of aged cells at different chronological age points (Day 1, 4, 7 and 10) with a microplate reader, where the outgrowth of different chronological age points was plotted relative to Day 1. All data are represented as mean±SD. **P<0.01 and ****P<0.0001 are based on two-way ANOVA, followed by Dunnett's multiple comparisons test. N.s. denotes non-significance.

FIG. 8 shows a photograph of outgrowth in 96-well plates with different concentrations of 2,5-AM at various chronological age points (Day 1, 4, 7 and 10) after incubation for 48 hours at 30° C. At each chronological age points, a 3 μL culture was spotted onto the YPD agar plate.

FIG. 9 refers to a graph evaluating cell growth at different concentrations of 2,5-AM between CEN.PK113-7D wild-type and snflΔ deletion strains. All data are represented as mean±SD. **P<0.01 and ****P<0.0001 are based on two-way ANOVA, followed by Sidak's multiple comparisons test. N.s. denotes non-significance.

FIG. 10 refers to graphs evaluating the effects of different concentrations of test agent on OD600 nm. [FIG. 10A] shows a graph evaluating the effects of different concentrations of 2,5-AM, [FIG. 10B] shows a graph evaluating the effects of different concentrations of fructose, [FIG. 10C] shows a graph evaluating the effects of different concentrations of mannitol, [FIG. 10D] shows a graph evaluating the effects of different concentrations of maltose, and [FIG. 10E] shows a graph evaluating the effects of different concentrations of sorbitol on OD600 nm at different time points (24 hours, 48 hours, and 72 hours). All data are represented as mean±SD.

FIG. 11 refers to graphs evaluating the effects of different concentrations of test agent on CLS. [FIG. 11A] shows a graph evaluating the effects of different concentrations of 2,5-AM, [FIG. 11B] shows a graph evaluating the effects of different concentrations of fructose, [FIG. 11C] shows a graph evaluating the effects of different concentrations of mannitol, [FIG. 11D] shows a graph evaluating the effects of different concentrations of maltose, and [FIG. 11E] shows a graph evaluating the effects of different concentrations of sorbitol on the CLS at different chronological age points. The growth time point 72 hours was considered as Day 1. All data are represented as mean±SD. *P<0.05, **P<0.01, and ****P<0.0001 are based on two-way ANOVA, followed by Dunnett's multiple comparisons test. N.s. denotes non-significance.

FIG. 12 refers to graphs evaluating the effect of different concentrations of test agent on outgrowth. [FIG. 12A] shows a graph evaluating the effects of different concentrations of 2,5-AM, [FIG. 12B] shows a graph evaluating the effects of different concentrations of fructose, [FIG. 12C] shows a graph evaluating the effects of different concentrations of mannitol, [FIG. 12D] shows a graph evaluating the effects of different concentrations of maltose, and [FIG. 12E] shows a graph evaluating the effects of different concentrations of sorbitol on CLS at different chronological age points using the outgrowth method in YPD liquid medium. The growth time point 72 hours was considered as Day 1. The outgrowth of different chronological age points was plotted relative to Day 1. All data are represented as mean±SD. ****P<0.0001 is based on two-way ANOVA, followed by Dunnett's multiple comparisons test. N.s. denotes non-significance.

FIG. 13 shows a photograph of outgrowth in YPD liquid medium of 96-well plates with different concentrations of 2,5-AM, fructose, mannitol, maltose, and sorbitol at various chronological age points after incubation for 24 hours at 30° C.

FIG. 14 shows a photograph of outgrowth on YPD agar plates with different concentrations of 2,5-AM, fructose, mannitol, maltose, and sorbitol at various chronological age points after incubation for 24 hours at 30° C.

FIG. 15 refers to testing the effects of 2,5-AM analogs on yeasts. [FIG. 15A] refers to a graph evaluating the effects of 2,5-AM analogs on CLS of yeasts. Cell survival was quantified at chronological age point Day 7, and the growth time point 72 hours was considered as Day 1. [FIG. 15B] refers to a graph evaluating the effect of 2,5-AM analogs on CLS using the outgrowth method in YPD liquid medium. The growth time point 72 hours was considered as Day 1, and the graph was plotted relative to Day 1.

FIG. 16 refers to the CLS extension of the yeast by sorbitol. [FIG. 16A] refers to graph evaluating the effects of different concentrations of sorbitol on cell growth (OD600 nm) at 72 hours. All data are represented as mean±SD. Data analysis is based on ordinary one-way ANOVA, followed by Dunnett's multiple comparisons test. N.s. denotes non-significance. [FIG. 16B] refers to a graph evaluating the effect of different concentrations of sorbitol on the CLS of aged cells. [FIG. 16C] refers to photographs of outgrowths at different chronological age points. All data are represented as mean±SD. **P<0.01 and ****P<0.0001 are based on ordinary two-way ANOVA, followed by Dunnett's multiple comparisons test. N.s. denotes non-significance.

FIG. 17 refers to a set of graphs showing the outcome of the CLS determination using the PI assay with 2,5-AM treatment. Cells were seeded in a 96-well plate with PI dissolved in the D10 medium. Fluorescence reading was taken on the sixth day after seeding. [FIG. 17A] is a graph showing the results for IMR90 cells and [FIG. 17B] is a graph showing the results for HEK293 cells.

FIG. 18 refers to a set of photographic images showing the CLS determination by qualitatively assessing the ability of the cells to proliferate. Cells were plated without (control) or with 2,5-AM compound treatment at several concentrations. On the designated day after the cells were seeded and treated, cells were trypsinized and 2% (4 μL out of 200 μL) of the cells were transferred to a 6-well assay plate with fresh D10 medium. The assay plate was incubated at 37° C. in an incubator with 5% CO₂ for seven days before staining with the Crystal Violet Assay. [FIG. 18A] shows the photographic image of the assay result after Day 4. [FIG. 18A] shows the photographic image of the assay result after Day 6. [FIG. 18A] shows the photographic image of the assay result after Day 8.

FIG. 19 refers to a graph evaluating the effect of different concentrations of 2,5-AM on the CLS of HEK293 cells via the outgrowth method using the PrestoBlue™ Cell Viability Reagent in a 96-well plate. Fluorescence reading of samples was measured at excitation and emission wavelengths of 560 nm and 590 nm, respectively. Cell survival on Day 4 was determined by normalization of outgrowth fluorescence intensity with Day 1. All data are represented as mean±SD. *P<0.05, **P<0.01, ***P<0.001, and ****P<0.0001 are based on ordinary one-way ANOVA, followed by Dunnett's multiple comparisons test. N.s. denotes non-significance.

FIG. 20 refers to a set of graphs showing the outcome of the CLS determination using the outgrowth assay with 2,5-AM treatment. Cells were seeded in a 96-well plate with PI dissolved in the D10 medium. Fluorescence reading was taken on the sixth day after seeding. [FIG. 20A] is a graph showing the results for IMR90 cells and [FIG. 20B] is a graph showing the results for HEK293 cells.

FIG. 21 refers to a set of graphs showing the HTS outcome of various chemicals to identify anti-aging compounds. [FIG. 21A] refers to a graph evaluating the CLS of different DMSO-soluble compounds using the PI method. [FIG. 21B] refers to a graph evaluating the CLS of different water-soluble compounds using the PI method. 50 nM rapamycin and 20 mM 2,5-AM were used as positive controls. The graphs depict the results from Day 6.

FIG. 22 refers to a set of graphs evaluating the effects of different concentrations of test agent on the CLS of the wild-type CEN.PK113-7D yeast strain in a 96-well plate. [FIG. 22A] shows a graph evaluating the effects of different concentrations of hemin, [FIG. 22B] shows a graph evaluating the effects of different concentrations of 5-fluorouracil, [FIG. 22C] shows a graph evaluating the effects of different concentrations of methotrexate, and [FIG. 22D] shows a graph evaluating the effects of different concentrations of atorvastatin. Aged cell survival was measured on different days relative to the outgrowth of Day 2.

FIG. 23 refers to the effects of different concentration of hemin on the lifespan of human cells, HEK293 and IMR90. [FIG. 23A] shows a set of photographic images that qualitatively assesses the proliferation of cells for CLS determination. Cells were incubated without (control) and with hemin supplementation at concentrations of 0, 12.5 and 25 μM. [FIG. 23B] shows a graph evaluating CLS in cells without (control) and with different concentrations of hemin using the PI method. [FIG. 23C] shows a graph assessing the proliferation of HEK293 cells without (control) and with hemin supplementation on Day 4 for CLS determination. [FIG. 23D] shows a graph assessing the proliferation of HEK293 cells without (control) and with hemin supplementation on Day 8 for CLS determination. [FIG. 23E] shows a graph assessing the proliferation of IMR90 cells without (control) and with hemin supplementation on Day 4 for CLS determination. [FIG. 23F] shows a graph assessing the proliferation of IMR90 cells without (control) and with hemin supplementation on Day 8 for CLS determination. CLS determination in FIGS. 23C to F was determined by the outgrowth method, using PrestoBlue™ Cell Viability Reagent in a 96-well plate.

### EXAMPLES

Non-limiting examples of the invention will be further described in greater detail by reference to specific Examples, which should not be construed as in any way limiting the scope of the invention.

### Materials

The prototrophic wild-type Saccharomyces cerevisiae CEN.PK113-7D yeast strain genetic background (Euroscarf, Köhlerweg, Oberursel, Germany), and the Saccharomyces cerevisiae BY4743 strain (Euroscarf, Köhlerweg, Oberursel, Germany) were used. The CEN.PK113-7D snflΔ deletion strain was constructed. The standard rich Yeast-Extract Peptone Dextrose (YPD) medium (1% w/v Bacto yeast extract, 2% w/v Bacto peptone, and 2% w/v glucose) (BD, Franklin Lakes, New Jersey, United States of America), YPD agar (2.5% w/v Bacto agar) (BD, Franklin Lakes, New Jersey, United States of America), and synthetic defined (SD) medium (BD, Franklin Lakes, New Jersey, United States of America) used here contained 6.7 g/L Difco™ Yeast Nitrogen Base (without amino acids and with ammonium sulfate) (BD, Franklin Lakes, New Jersey, United States) and 2% w/v glucose. Rapamycin (Enzo Life Sciences, Farmingdale, New York, United States) stock solution was prepared in dimethyl sulfoxide (DMSO) (Sigma-Aldrich, St. Louis, Missouri, United States of America). The working concentrations of 2,5-anhydro-D-mannitol (Santa Cruz Biotechnology, Dallas, Texas, United States), D-fructose (Sigma-Aldrich, St. Louis, Missouri, United States of America), and D-mannitol (Sigma-Aldrich, St. Louis, Missouri, United States of America), D-maltose (Sigma-Aldrich, St. Louis, Missouri, United States of America) and D-sorbitol (Sigma-Aldrich, St. Louis, Missouri, United States of America) were freshly prepared by direct dissolution of the powder in medium and filter sterilization. Hemin (Sigma-Aldrich, St. Louis, Missouri, United States of America), 5-fluorouracil (Sigma-Aldrich, St. Louis, Missouri, United States of America), atorvastatin (Sigma-Aldrich, St. Louis, Missouri, United States of America), and methotrexate (Sigma-Aldrich, St. Louis, Missouri, United States of America) stock solutions were prepared in DMSO, followed by serial dilution to form various concentrations. The final concentration of DMSO did not exceed 1% v/v in any assay for yeast experiments. The final DMSO concentration was kept at 0.05% v/v in all human cell line experiments.

The various compounds tested in Table 1 were obtained from Sigma-Aldrich (St. Louis, Missouri, United States of America), MedChemExpress (Monmouth Junction, New Jersey, United States of America), Enzo Life Sciences, Inc. (Farmingdale, New York, United States of America), Santa Cruz Biotechnology (Dallas, Texas, United States of America), or Biosynth Carbosynth (Compton, Berkshire, United Kingdom).

### Methods

### Yeast Growth Conditions

Yeast strain was recovered from frozen glycerol stock at 30° C. on YPD agar medium. Yeast was left to propagate in SD medium overnight at 30° C. with shaking at 220 revolutions per minute (rpm). Cells propagated overnight were diluted to a starting optical density at 600 nm (OD₆₀₀) of approximately 0.2 in fresh SD medium to begin the chronological lifespan (CLS) experiments.

### Chronological Lifespan Analysis in Yeast

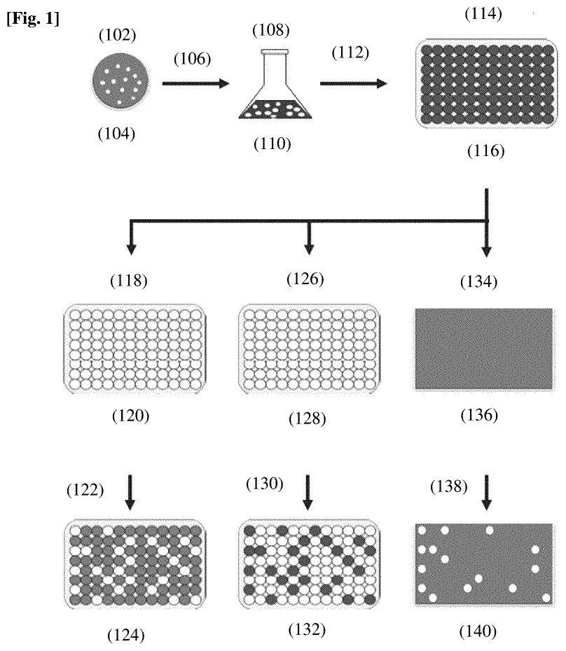

CLS experiments were performed in 96-well plates with 200 μL yeast culture. Cellular inoculums were transferred into 96-well plates containing serially double-diluted concentrations (0 to 10 nM) of rapamycin. Cellular inoculum was transferred into 96-well plates containing serially double-diluted concentrations (0 to 8 mM) of 2,5-anhydro-D-mannitol, D-fructose, D-mannitol, D-maltose, and D-sorbitol in SD medium. Cells were incubated at 30° C., and the growth was measured at different time points. The growth time point 72 hours was considered as Day 1 for the CLS assay. Cell survival was quantified at various age time points by three different approaches: (i) PI fluorescence-based method, (ii) outgrowth in YPD liquid medium, and (iii) spotting assay (FIG. 1).

### Propidium Iodide Fluorescence-Based Method in Yeast

Yeast cells (40 μL) from different age time points were transferred into a second 96-well plate (different to the 96-well plate in which the CLS analysis as outlined above was performed). Cells were washed and incubated in 100 μL 1×phosphate buffered saline (PBS) with propidium iodide (PI) (5 μg/mL) for 15 minutes in the dark. Positive and negative control samples were included for quantitative analysis. Positive control (cells boiled at 100° C. for 15 minutes) was PI-stained and processed in the same 96-well plate. Samples without PI-stained cells served as the negative control. After incubation, cells were washed and resuspended in 100 μL PBS. The fluorescence reading of the samples (excitation and emission wavelengths at 535 nm and 617 nm, respectively) and OD₆₀₀ were measured with a microplate reader (BioTek, Winooski, Vermont, United States). The fluorescence intensity of each sample was normalized with OD₆₀₀. The background fluorescence signal of the unstained negative sample was subtracted from the normalized fluorescence intensity of each sample. The obtained fluorescence intensity of the positive control (boiled dead cells) was considered 0% cell survival. Cell death was validated by allowing the boiled dead cells to grow in medium. Cell survival at different age time points of samples were calculated by normalizing the fluorescence intensity with positive control sample (boiled cells).

### Cell survival was calculated using the formula:

$$Survival = ( 1 - \frac{\frac{{I}_{ij} ( 535 nm / 617 nm )}{{OD}_{ij} ( 600 nm )} - \frac{{I}_{c} ( 535 nm / 617 nm )}{{OD}_{c} ( 600 nm )}}{\frac{{I}_{D} ( 535 nm / 617 nm )}{{OD}_{D} ( 600 nm )} - \frac{{I}_{c} ( 535 nm / 617 nm )}{{OD}_{c} ( 600 nm )}} ) 100 \%$$

where Iᵢⱼ(535 nm/617 nm) is the fluorescence intensity (excitation and emission wavelengths of 535 nm and 617 nm, respectively) measured at each microplate well with plate coordinates i and j, I_{D}(535 nm/617 nm) is the fluorescence intensity measured for a well with 100% dead cells stained with PI, I_{C}(535 nm/617 nm) is the fluorescence intensity recorded for cells without PI, and the respective OD values are the absorption measured for the respective cells at 600 nm, normalizing for the amount of cells.

### Yeast Outgrowth in YPD Liquid Medium

Yeast stationary culture (3 μL) of different age time points were transferred to a second 96-well plate, containing 200 μL YPD medium, and incubated for 24 hours at 30° C. Outgrowth (OD₆₀₀) of aged cells was measured by the microplate reader. Quantification of cell survival for each age point was determined as relative to Day 1 (considered 100% cell survival). This quantification was performed by using the formula,

$$Cell survival on Day n = \frac{{Outgrowth}_{Day n}}{{Outgrowth}_{Day 1}} \times 100$$

where Outgrowth_{Day n} refers to the OD600 nm absorbance reading recorded on a specific day, Outgrowth_{Day 1} refers to the OD600 nm absorbance reading recorded on the first day where 100% cell survival is assumed.

### Yeast Spotting Assay

Yeast stationary culture (3 μL) of different age time points were spotted onto a YPD agar plate and incubated for 48 hours at 30° C. The outgrowth of aged cells on the YPD agar plate was photographed using the Bio-Rad GelDoc Imaging System (Bio-Rad, Hercules, California, United States).

### High-Throughput Screening

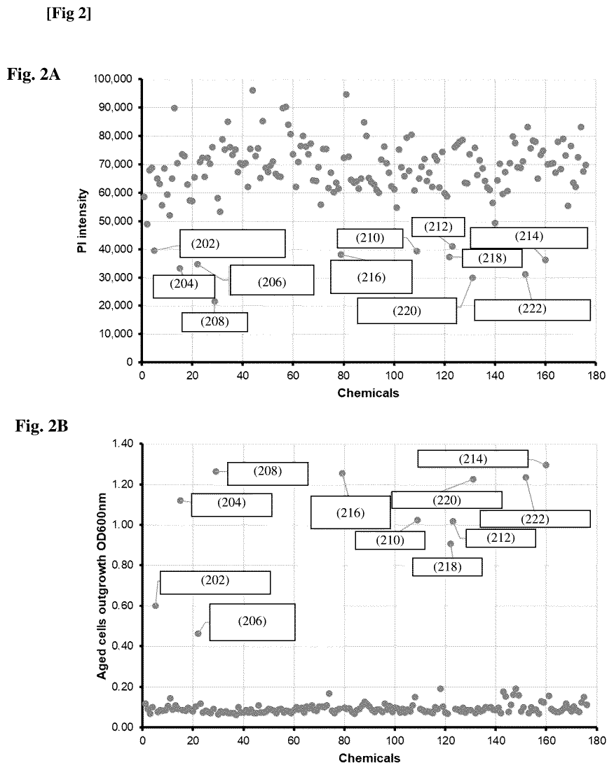

High-throughput screening of various chemicals was tested using the same protocol for the propidium iodide fluorescence-based method as described above. The prototrophic yeast strain (CEN.PK113-7D) was grown in the synthetic defined medium with different chemical reagents at their respective single testing concentration (Table 1) in addition to eight replicates of DMSO and water controls in 96-well plates at 30° C. Cell survival at age point day 7 was quantified, and the growth time point 72 hours was considered day 1 (FIG. 2A).

The CLS of the aged cells was further determined by the yeast outgrowth in YPD liquid medium method as described above. The growth time point 72 hours was considered as day 1. At chronological age point day 14, 3-μL of culture were transferred to a second 96-well plate containing 200 μL YPD medium. Outgrowth OD600 nm in YPD liquid medium was measured after incubation for 24 hours at 30° C. using a microplate reader (FIG. 2B).

### Human Cell Culture

Human embryonic kidney 293 (HEK293) cells (ATCC, Manassas, Virginia, United States of America), lung epithelial (A549) cells (ATCC, Manassas, Virginia, United States of America) and fibroblast (IMR90) cells (ATCC, Manassas, Virginia, United States of America) were cultured in the standard D10 medium, consisting of high-glucose DMEM (HyClone, Cytiva, Marlborough, Massachusetts, USA) supplemented with 10% Fetal Bovine Serum (FBS) (Gibco™, ThermoFisher Scientific, Waltham, Massachusetts, USA) and 1% Penicillin Streptomycin Solution (Gibco™, ThermoFisher Scientific, Waltham, Massachusetts, USA). All cells were cultured in a humidified incubator with 5% CO₂ at 37° C.

### Chemical Treatment of Human Cell Culture

The stock solution of rapamycin, hemin, 5-fluorouracil, atorvastatin, and methotrexate was prepared in DMSO, followed by serial dilution to form various concentrations used in the experiment. The final DMSO concentration was kept at 0.05% in all experiments. The stock solution of 2,5-anhydro-D-mannitol was prepared in D10 medium. The chemical library used in screening were tested at 1 μM concentrations. Controls used were the same as the solvent applied, DMSO or water.

### Chronological Lifespan Analysis in Human Cells Using Crystal Violet

80,000 cells were seeded in a 96-well culture plate with or without compound treatment. On Day 4, Day 6, and Day 8 after seeding, floating dead cells were aspirated with exhausted D10 medium. Then, attached cells were gently washed with 1×PBS to remove D10 medium residue and treated with 0.2 mL of 0.25% trypsin. Cells were gently mixed by pipetting and a 2% aliquot (4 μL) was transferred to 6-well experiment plates with fresh D10 medium. After seven days of growth in a 37° C. incubator with 5% CO₂, the crystal violet assay was performed on the test plate to determine the viability of cultured cells. Specifically, the media was aspirated, and the cells were stained with 0.05% w/v crystal violet, followed by shaking at an oscillation speed of 20 for 20 minutes. The crystal violet solution was aspirated, the cells were washed once with water, and the water was aspirated. The plate was inverted and tapped dried to remove residual water, then air dried for at least 2 hours before the plates were imaged using a scanner (Epson Perfection V700 photo, Epson, Singapore).

### Chronological Lifespan Analysis in Human Cells Using PrestoBlue™ Cell Viability Reagent

The seeding, washing and trypsinization process was performed similarly to that for the crystal violet assay. 80,000 cells were seeded in a 96-well culture plate with or without compound treatment. At different age time points after seeding, floating dead cells were aspirated with exhausted D10 medium, cells were gently washed with 1×PBS to remove D10 medium residue and treated with 0.2 mL of 0.25% trypsin. Cells were gently mixed by pipetting and a 5% aliquot (10 μL) or 10% aliquot (20 μL) were transferred to 96-well experiment plates with fresh D10 medium. After one day of growth in a 37° C. incubator with 5% CO₂, the cell number was assessed with PrestoBlue™ Cell Viability Reagent (Invitrogen™, Waltham, Massachusetts, USA) according to the manufacturer's protocol. In brief, the cell viability reagent was diluted 10-fold with growth D10 medium. The D10 medium was aspirated from the 96-well plate and replaced with the diluted PrestoBlue reagent. After incubation in a 37° C. incubator with 5% CO₂ for 3 to 4 hours, reading was taken with a BioTek Synergy MX microplate reader (BioTek, Winooski, Vermont, United States) at excitation and emission wavelengths of 560 nm and 590 nm, respectively.

### Chronological Lifespan Analysis in Human Cells Using Propidium Iodide Fluorescence-Based Method

The assay was performed by preparing the cells with PI (2 μg/mL or 5 μg/mL) in a D10 medium. 200 μL culture (8×104 cells) was seeded per well in a 96-well plate with different concentrations of the anti-aging drug (rapamycin, 2,5-AM, plicamycin, copanlisib dihydrochloride, nedaplatin, hemin, 5-fluorouracil, methotrexate, or atorvastatin) and no treatment control (DMSO). The culture plate was kept away from light throughout the experiment and incubated at 37° C. with 5% CO₂. At different age time points, reading was taken with a Synergy MX microplate reader (BioTek, Winooski, Vermont, United States) at 535 nm excitation and 617 nm emission.

### Data Analysis

Statistical analysis of all the results such as mean value, standard deviations, and correlation (R²), significance and graphing were performed using the GraphPad Prism v.9.3.1 and v.9.4.1 software. The results were statistically compared using one-way and two-way Analysis of Variance (ANOVA) tests, followed by multiple comparison by Dunnett's or Sidak's post hoc test. In all the graph plots, *P<0.05, **P<0.01, *** P<0.001, and ****P<0.0001 indicated significance, while n.s. indicated non-significance.

### Example 1: Screening and Identification of 2,5-anhydro-D-mannitol as a Novel Anti-Aging Compound

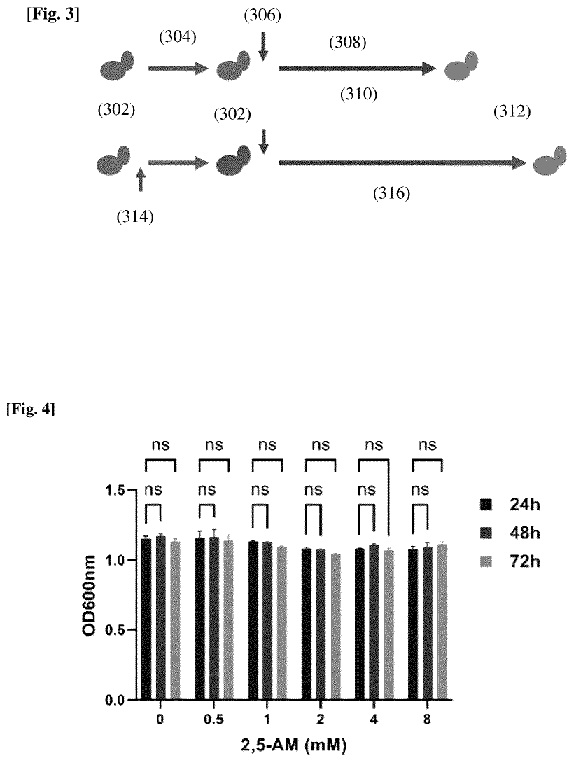

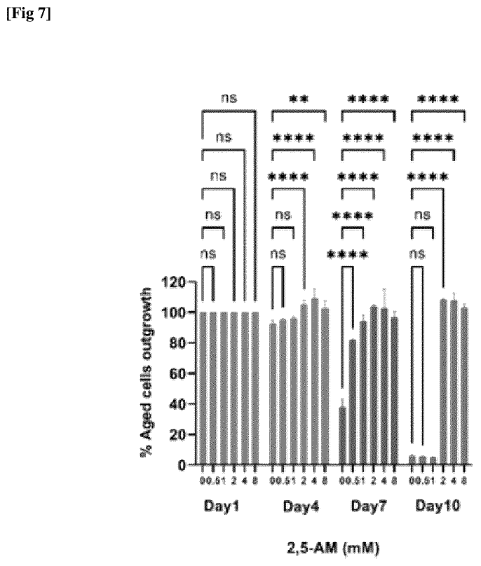

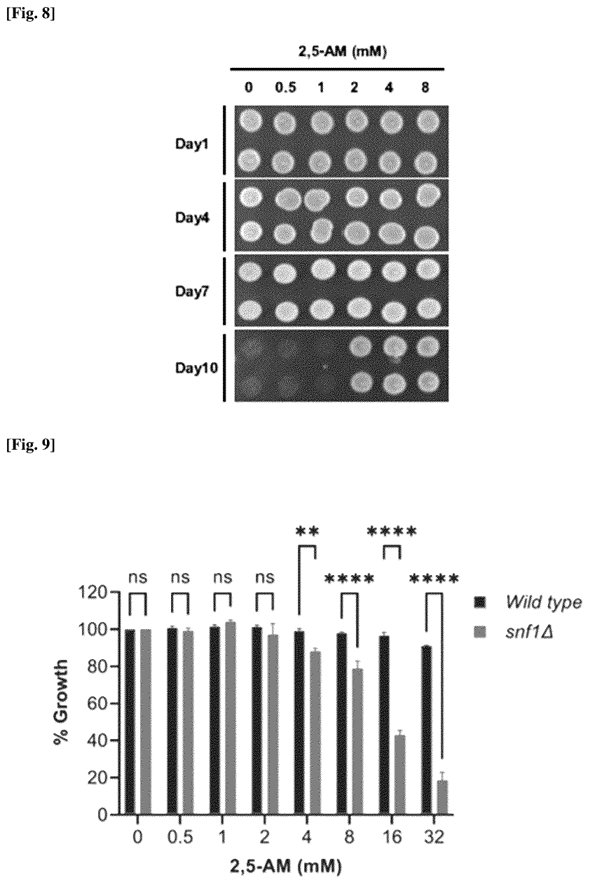

Hundreds of chemical agents were screened for the identification of anti-aging compounds (FIGS. 2A and 2B; Table 1) that may extend the CLS of yeasts (FIG. 3) using the PI method. Of these, the compound 2,5-anhydro-D-mannitol (2,5-AM) was surprisingly shown to extend the CLS of yeasts. FIGS. 4 to 8 depict the outcome of the detailed follow-up experiments for this compound. The anti-aging activity of 2,5-AM was tested at different concentrations on the 96-well plate.

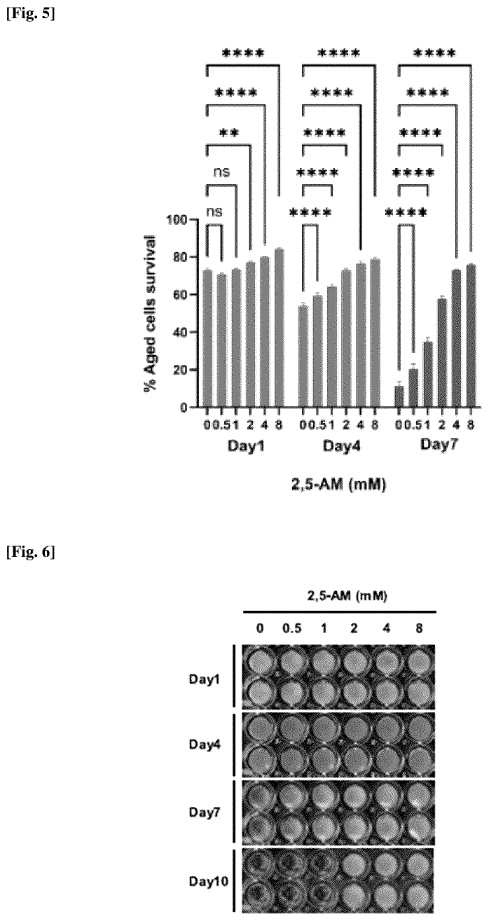

Firstly, the effect of 2,5-AM on cell growth was determined. Yeast cells were incubated with varying concentrations of 2,5-AM in the SD medium. Cell growth was measured at different time points (24 hours, 48 hours, and 72 hours). Cell growth reached the same saturation level after 24 hours for all tested concentrations of 2,5-AM (FIG. 4). Then, the survival of chronologically aged cells supplemented with different concentrations of 2,5-AM was measured using the PI fluorescence method. At each chronological age point, a 3 μL culture was transferred to a second 96-well plate containing 200 μL YPD medium. The 72-hour growth culture was considered Day 1 for the CLS analysis. The viability fraction was calculated, and a survival graph was plotted for different chronological age time points (FIG. 5). It was found that 2,5-AM extended the CLS in a concentration-dependent manner. The survival of aged cells supplemented with 2,5-AM (4 mM and 8 mM) on Day 4 was approximately 80%. However, the survival of aged cells without 2,5-AM was approximately 50%. On Day 7, survival of 2,5-AM supplemented aged cells was approximately 75%. However, the survival of aged cells without 2,5-AM was reduced to less than 20%. The antiaging activity of 2,5-AM was also verified by outgrowth assays (FIGS. 6 to 8).

TABLE 1
List of chemicals screened.

| | | | Concentration | | PI | | Yeast CLS |
|---|---|---|---|---|---|---|---|
| Number | Chemical name | Solvent | tested | | intensity | Outgrowth | increase |
| 1 | Aucubin | DMSO | 100 | μM | 58,414.0 | 0.1 | No |
| 2 | D-Glucuronic acid | DMSO | 100 | μM | 48,839.6 | 0.1 | No |
| 3 | β-Lapachone | DMSO | 100 | μM | 67,892.9 | 0.1 | No |
| 4 | Isocytosine | DMSO | 100 | μM | 68,813.6 | 0.1 | No |
| 5 | Rifamycin (sodium) | DMSO | 100 | μM | 39,446.0 | 0.6 | Yes |
| 6 | Cryptochlorogenic acid | DMSO | 100 | μM | 65,040.0 | 0.1 | No |
| 7 | Isochlorogenic acid A | DMSO | 100 | μM | 63,157.7 | 0.1 | No |
| 8 | Rhynchophylline | DMSO | 100 | μM | 55,562.5 | 0.1 | No |
| 9 | Mupirocin | DMSO | 100 | μM | 68,477.7 | 0.1 | No |
| 10 | 3,5-Dihydroxybenzoic acid | DMSO | 100 | μM | 59,369.7 | 0.1 | No |
| 11 | Ethylparaben | DMSO | 100 | μM | 52,097.7 | 0.1 | No |
| 12 | Nudifloramide | DMSO | 100 | μM | 65,023.3 | 0.1 | No |
| 13 | 4-Methoxycinnamic acid | DMSO | 100 | μM | 89,890.5 | 0.1 | No |
| 14 | Cefoselis (sulfate) | DMSO | 100 | μM | 70,425.6 | 0.1 | No |
| 15 | DL-Serine | DMSO | 100 | μM | 33,155.7 | 1.1 | Yes |
| 16 | Avibactam (sodium) | DMSO | 100 | μM | 73,562.3 | 0.1 | No |
| 17 | (S)-2-Hydroxy-3- | DMSO | 100 | μM | 72,969.5 | 0.1 | No |
| | phenylpropanoic acid | | | | | | |
| 18 | Vincristine (sulfate) | DMSO | 100 | μM | 62,812.1 | 0.1 | No |
| 19 | N-Acetyl-L-tyrosine | DMSO | 100 | μM | 57,322.5 | 0.1 | No |
| 20 | Rubitecan | DMSO | 100 | μM | 57,075.3 | 0.1 | No |
| 21 | Royal Jelly acid | DMSO | 100 | μM | 65,406.1 | 0.1 | No |
| 22 | β-Estradiol 17-acetate | DMSO | 100 | μM | 34,621.0 | 0.5 | Yes |
| 23 | 3,4-Dimethoxycinnamic | DMSO | 100 | μM | 70,914.2 | 0.1 | No |
| | acid | | | | | | |
| 24 | 2-Naphthol | DMSO | 100 | μM | 72,298.9 | 0.1 | No |
| 25 | N2,N2-Dimethylguanosine | DMSO | 100 | μM | 65,619.9 | 0.1 | No |
| 26 | Proxyphylline | DMSO | 100 | μM | 72,232.1 | 0.1 | No |
| 27 | Amarogentin | DMSO | 100 | μM | 70,083.5 | 0.1 | No |
| 28 | Lappaconitine | DMSO | 100 | μM | 76,000.0 | 0.1 | No |
| | (hydrobromide) | | | | | | |
| 29 | Rapamycin | DMSO | 100 | μM | 21,460.5 | 1.3 | Yes |
| 30 | 4-Ethylphenol | DMSO | 100 | μM | 58,041.7 | 0.1 | No |
| 31 | Purine | DMSO | 100 | μM | 53,185.1 | 0.1 | No |
| 32 | (S)-Indoximod | DMSO | 100 | μM | 78,648.2 | 0.1 | No |
| 33 | Actinonin | DMSO | 100 | μM | 75,254.2 | 0.1 | No |
| 34 | Cirsimaritin | DMSO | 100 | μM | 85,017.1 | 0.1 | No |
| 35 | 6-Biopterin | DMSO | 100 | μM | 75,974.5 | 0.1 | No |
| 36 | Hederagenin | DMSO | 100 | μM | 73,281.0 | 0.1 | No |
| 37 | Pentadecanoic acid | DMSO | 100 | μM | 75,173.9 | 0.1 | No |
| 38 | Staurosporine | DMSO | 100 | μM | 67,250.9 | 0.1 | No |
| 39 | Xanthoxylin | DMSO | 100 | μM | 70,352.1 | 0.1 | No |
| 40 | D-(+)-Cellobiose | DMSO | 100 | μM | 69,857.4 | 0.1 | No |
| 41 | Escin | DMSO | 100 | μM | 70,495.1 | 0.1 | No |
| 42 | 5α-Cholestan-3-one | DMSO | 100 | μM | 62,022.6 | 0.1 | No |
| 43 | 20(S)-Hydroxycholesterol | DMSO | 100 | μM | 75,450.9 | 0.1 | No |
| 44 | Anthraquinone-2-carboxylic | DMSO | 100 | μM | 96,099.2 | 0.1 | No |
| | acid | | | | | | |
| 45 | L-Kynurenine | DMSO | 100 | μM | 72,824.1 | 0.1 | No |
| 46 | Quinine (hydrochloride | DMSO | 100 | μM | 75,648.7 | 0.1 | No |
| | dihydrate) | | | | | | |
| 47 | DL-3-Phenyllactic acid | DMSO | 100 | μM | 65,266.9 | 0.1 | No |
| 48 | Retinoic acid | DMSO | 100 | μM | 85,242.6 | 0.1 | No |
| 49 | Schisantherin B | DMSO | 100 | μM | 68,765.7 | 0.1 | No |
| 50 | Narciclasine | DMSO | 100 | μM | 67,161.8 | 0.1 | No |
| 51 | Doripenem (monohydrate) | DMSO | 100 | μM | 69,608.7 | 0.1 | No |
| 52 | Diflorasone | DMSO | 100 | μM | 71,051.3 | 0.1 | No |
| 53 | Chlorogenic acid | DMSO | 100 | μM | 66,593.5 | 0.1 | No |
| 54 | Glucosamine | DMSO | 100 | μM | 65,905.5 | 0.1 | No |
| | (hydrochloride) | | | | | | |
| 55 | Gardenoside | DMSO | 100 | μM | 65,525.5 | 0.1 | No |
| 56 | Aloin B | DMSO | 100 | μM | 89,712.0 | 0.1 | No |
| 57 | 4-Methylumbelliferone | DMSO | 100 | μM | 90,234.6 | 0.1 | No |
| 58 | Lanolin | DMSO | 100 | μM | 84,056.6 | 0.1 | No |
| 59 | (R)-pyrrolidine-2-carboxylic | DMSO | 100 | μM | 80,551.7 | 0.1 | No |
| | acid | | | | | | |
| 60 | Glycodeoxycholic acid | DMSO | 100 | μM | 73,459.3 | 0.1 | No |
| | (monohydrate) | | | | | | |
| 61 | (−)-Epigallocatechin | DMSO | 100 | μM | 62,025.6 | 0.1 | No |
| 62 | 2′-Deoxyadenosine | DMSO | 100 | μM | 70,796.4 | 0.1 | No |
| 63 | Wogonin | DMSO | 100 | μM | 76,348.2 | 0.1 | No |
| 64 | Schisandrol B | DMSO | 100 | μM | 80,051.7 | 0.1 | No |
| 65 | 2,4-Dihydroxybenzoic acid | DMSO | 100 | μM | 76,326.3 | 0.1 | No |
| 66 | Hederacoside C | DMSO | 100 | μM | 73,515.9 | 0.1 | No |
| 67 | Noricaritin | DMSO | 100 | μM | 77,179.8 | 0.1 | No |
| 68 | Canrenone | DMSO | 100 | μM | 64,295.2 | 0.1 | No |
| 69 | Glycoursodeoxycholic acid | DMSO | 100 | μM | 64,205.2 | 0.1 | No |
| 70 | Glycodeoxycholic Acid | DMSO | 100 | μM | 68,981.6 | 0.1 | No |
| 71 | Cilastatin | DMSO | 100 | μM | 55,728.9 | 0.1 | No |
| 72 | Emetine (dihydrochloride | DMSO | 100 | μM | 75,341.5 | 0.1 | No |
| | hydrate) | | | | | | |
| 73 | Norgestrel | DMSO | 100 | μM | 75,338.8 | 0.1 | No |
| 74 | Heterophyllin B | DMSO | 100 | μM | 61,587.8 | 0.2 | No |
| 75 | Hispidin | DMSO | 100 | μM | 67,098.8 | 0.1 | No |
| 76 | Cinnamic acid | DMSO | 100 | μM | 60,256.3 | 0.1 | No |
| 77 | Catechin | DMSO | 100 | μM | 63,589.5 | 0.1 | No |
| 78 | [6]-Gingerol | DMSO | 100 | μM | 61,395.3 | 0.1 | No |
| 79 | Epiberberine (chloride) | DMSO | 100 | μM | 38,027.2 | 1.3 | Yes |
| 80 | Terconazole | DMSO | 100 | μM | 72,350.0 | 0.1 | No |
| 81 | Isoalantolactone | DMSO | 100 | μM | 94,566.8 | 0.1 | No |
| 82 | 2-Furoic acid | DMSO | 100 | μM | 72,748.2 | 0.1 | No |
| 83 | Vanillic acid | DMSO | 100 | μM | 64,546.4 | 0.1 | No |
| 84 | 5-Acetylsalicylic acid | DMSO | 100 | μM | 63,538.6 | 0.1 | No |
| 85 | Guggulsterone | DMSO | 100 | μM | 63,881.9 | 0.1 | No |
| 86 | Thymol | DMSO | 100 | μM | 61,503.8 | 0.1 | No |
| 87 | Homovanillic acid | DMSO | 100 | μM | 65,033.1 | 0.1 | No |
| 88 | Uridine | DMSO | 100 | μM | 84,894.9 | 0.1 | No |
| 89 | Gramicidin | DMSO | 100 | μM | 79,922.3 | 0.1 | No |
| 90 | Gossypol | DMSO | 100 | μM | 65,220.1 | 0.1 | No |
| 91 | Cyanidin-3-O-galactoside | DMSO | 100 | μM | 63,761.1 | 0.1 | No |
| | (chloride) | | | | | | |
| 92 | Kaempferol 3-O-β-D- | DMSO | 100 | μM | 62,874.6 | 0.1 | No |
| | glucuronide | | | | | | |
| 93 | L-Homoserine | DMSO | 100 | μM | 60,922.1 | 0.1 | No |
| 94 | D-Ribose(mixture of | DMSO | 100 | μM | 60,023.1 | 0.1 | No |
| | isomers) | | | | | | |
| 95 | Harmane | DMSO | 100 | μM | 71,690.9 | 0.1 | No |
| 96 | Geraniol | DMSO | 100 | μM | 76,340.1 | 0.1 | No |
| 97 | 3,4-Dimethoxyphenylacetic | DMSO | 100 | μM | 70,394.7 | 0.1 | No |
| | acid | | | | | | |
| 98 | 2-Phenylpropionic acid | DMSO | 100 | μM | 66,998.4 | 0.1 | No |
| 99 | Deltonin | DMSO | 100 | μM | 62,357.6 | 0.1 | No |
| 100 | 2-Oxovaleric acid | DMSO | 100 | μM | 61,246.4 | 0.1 | No |
| 101 | Estrone sulfate (potassium) | DMSO | 100 | μM | 54,751.8 | 0.1 | No |
| 102 | (R)-Citronellol | DMSO | 100 | μM | 75,290.6 | 0.1 | No |
| 103 | Euphorbia Factor L2 | DMSO | 100 | μM | 68,871.8 | 0.1 | No |
| 104 | β-Aminopropionitrile | DMSO | 100 | μM | 65,865.6 | 0.1 | No |
| 105 | Bronopol | DMSO | 100 | μM | 79,284.9 | 0.1 | No |
| 106 | Lincomycin (hydrochloride | DMSO | 100 | μM | 67,751.3 | 0.1 | No |
| | monohydrate) | | | | | | |
| 107 | Tridecanedioic acid | DMSO | 100 | μM | 80,407.7 | 0.1 | No |
| 108 | Calcifediol | DMSO | 100 | μM | 60,747.4 | 0.2 | No |
| 109 | Melanin | DMSO | 100 | μM | 39,360.1 | 1.0 | Yes |
| 110 | Methyl acetylacetate | DMSO | 100 | μM | 64,956.3 | 0.1 | No |
| 111 | L-Cysteine (hydrochloride | DMSO | 100 | μM | 69,216.4 | 0.1 | No |
| | hydrate) | | | | | | |
| 112 | Impulsin | DMSO | 100 | μM | 71,903.7 | 0.1 | No |
| 113 | Formamide | DMSO | 100 | μM | 64,135.2 | 0.1 | No |
| 114 | 3-Chloro-L-tyrosine | DMSO | 100 | μM | 67,122.0 | 0.1 | No |
| 115 | Tetramethylpyrazine | DMSO | 100 | μM | 62,003.6 | 0.1 | No |
| 116 | Esculin | DMSO | 100 | μM | 72,890.7 | 0.1 | No |
| 117 | DL-Panthenol | DMSO | 100 | μM | 71,468.6 | 0.1 | No |
| 118 | Baimaside | DMSO | 100 | μM | 61,895.7 | 0.2 | No |
| 119 | Benzyl acetate | DMSO | 100 | μM | 74,321.9 | 0.1 | No |
| 120 | Gentiopicroside | DMSO | 100 | μM | 59,804.7 | 0.1 | No |
| 121 | Guaiazulene | DMSO | 100 | μM | 58,776.1 | 0.1 | No |
| 122 | Engeletin | DMSO | 100 | μM | 37,144.4 | 0.9 | Yes |
| 123 | Acivicin | DMSO | 100 | μM | 41,066.2 | 1.0 | Yes |
| 124 | Tenofovir (hydrate) | Water | 100 | μM | 76,065.0 | 0.1 | No |
| 125 | Metyrosine | Water | 100 | μM | 76,947.2 | 0.1 | No |
| 126 | Ripasudil | Water | 100 | μM | 77,962.0 | 0.1 | No |
| 127 | Diphenmanil (methylsulfate) | Water | 100 | μM | 78,623.4 | 0.1 | No |
| 128 | Ambroxol (hydrochloride) | Water | 100 | μM | 63,509.0 | 0.1 | No |
| 129 | VAL-083 | Water | 100 | μM | 63,325.5 | 0.1 | No |
| 130 | Aliskiren (hemiumarate) | Water | 100 | μM | 73,607.3 | 0.1 | No |
| 131 | Selenomethionine | Water | 100 | μM | 29,837.8 | 1.2 | Yes |
| 132 | Cisatracurium (besylate) | Water | 100 | μM | 75,862.3 | 0.1 | No |
| 133 | DL-Methionine | Water | 100 | μM | 66,281.7 | 0.1 | No |
| | methylsulfonium (chloride) | | | | | | |
| 134 | Biapenem | Water | 100 | μM | 71,409.3 | 0.1 | No |
| 135 | Dipotassium glycyrrhizinate | Water | 100 | μM | 68,519.8 | 0.1 | No |
| 136 | L-Ornithine (hydrochloride) | Water | 100 | μM | 64,179.1 | 0.1 | No |
| 137 | Fosphenytoin (disodium) | Water | 100 | μM | 61,860.0 | 0.1 | No |
| 138 | (R)-Baclofen | Water | 100 | μM | 61,049.3 | 0.1 | No |
| 139 | Trapidil | Water | 100 | μM | 56,443.9 | 0.1 | No |
| 140 | Xanthinol Nicotinate | Water | 100 | μM | 49,233.6 | 0.1 | No |
| 141 | Cerivastatin (sodium) | Water | 100 | μM | 64,250.5 | 0.1 | No |
| 142 | Aprotinin | Water | 100 | μM | 70,144.6 | 0.1 | No |
| 143 | Flavin adenine dinucleotide | Water | 100 | μM | 59,508.7 | 0.2 | No |
| | (disodium salt) | | | | | | |
| 144 | Biperiden (Hydrochloride) | Water | 100 | μM | 67,178.1 | 0.2 | No |
| 145 | Streptomycin (sulfate) | Water | 100 | μM | 60,638.5 | 0.1 | No |
| 146 | Cefadroxil | Water | 100 | μM | 70,326.1 | 0.1 | No |
| 147 | Diclofenac (Sodium) | Water | 100 | μM | 79,704.3 | 0.2 | No |
| 148 | Estramustine (phosphate | Water | 100 | μM | 77,514.0 | 0.2 | No |
| | sodium) | | | | | | |
| 149 | Amifostine | Water | 100 | μM | 69,013.2 | 0.2 | No |
| 150 | Quetiapine sulfoxide | Water | 100 | μM | 68,761.6 | 0.1 | No |
| | (dihydrochloride) | | | | | | |
| 151 | Minocycline (hydrochloride) | Water | 100 | μM | 71,078.2 | 0.1 | No |
| 152 | L-SelenoMethionine | Water | 100 | μM | 31,240.0 | 1.2 | Yes |
| 153 | Cangrelor (tetrasodium) | Water | 100 | μM | 83,173.6 | 0.1 | No |
| 154 | Chondroitin (sulfate) | Water | 30 | μg/mL | 75,572.6 | 0.1 | No |
| 155 | Hyaluronidase | Water | 30 | μg/mL | 78,406.7 | 0.1 | No |
| 156 | Heparin (Lithium salt) | Water | 30 | μg/mL | 77,829.1 | 0.1 | No |
| 157 | Heparin (sodium salt) | Water | 30 | μg/mL | 64,969.0 | 0.1 | No |
| | (MW 15 kDa) | | | | | | |
| 158 | Gentamicin (sulfate) | Water | 30 | μg/mL | 73,296.2 | 0.1 | No |
| 159 | Polidocanol | Water | 30 | μg/mL | 74,745.5 | 0.1 | No |
| 160 | 2,5-anhydromannitol | Water | 300 | μg/mL | 36,240.0 | 1.3 | Yes |
| 161 | DMSO control 1 | DMSO | | | 69,929.1 | 0.2 | |
| 162 | DMSO control 2 | DMSO | | | 70,278.0 | 0.1 | |
| 163 | DMSO control 3 | DMSO | | | 70,494.8 | 0.1 | |
| 164 | DMSO control 4 | DMSO | | | 67,035.1 | 0.1 | |
| 165 | DMSO control 5 | DMSO | | | 78,011.0 | 0.1 | |
| 166 | DMSO control 6 | DMSO | | | 68,377.5 | 0.1 | |
| 167 | DMSO control 7 | DMSO | | | 78,899.7 | 0.1 | |
| 168 | DMSO control 8 | DMSO | | | 73,013.1 | 0.1 | |
| 169 | Water control 1 | Water | | | 55,358.7 | 0.1 | |
| 170 | Water control 2 | Water | | | 76,405.5 | 0.1 | |
| 171 | Water control 3 | Water | | | 63,579.4 | 0.1 | |
| 172 | Water control 4 | Water | | | 62,094.3 | 0.1 | |
| 173 | Water control 5 | Water | | | 72,575.8 | 0.1 | |
| 174 | Water control 6 | Water | | | 83,192.6 | 0.1 | |
| 175 | Water control 7 | Water | | | 67,575.6 | 0.2 | |
| 176 | Water control 8 | Water | | | 69,669.4 | 0.1 | |

### Example 2: Anti-Aging Mechanism of 2,5-anhydro-D-mannitol

2,5-AM is a sugar molecule that can enter the glycolysis pathway. Glycolysis is a metabolic process in which glucose is first phosphorylated by hexokinase to form glucose-6-phosphate (G6P). Phosphoglucoisomerase interconverts G6P to fructose-6-phosphate (F6P) that is further metabolized into different downstream glycolytic intermediates, including pyruvate that enters the mitochondrial TCA cycle. 2,5-AM gets hydrolyzed only at upstream glycolytic steps that causes the accumulation of nonmetabolized 2,5-AM-1,6-bisphosphate (2,5-AMBP) in the cells. SNFl is a cellular energy sensor and highly conserved AMP-activated protein kinase (AMPK) in eukaryotes. Yeast cells that lack AMPK activity are associated with a hypersensitive growth phenotype in the presence of non-metabolized glycolytic intermediates.

To test whether 2,5-AM at similar concentrations to the CLS experiment would be processed by glycolysis enzymes, the growth sensitivity of the SNFl gene deletion strain was examined. The growth of the snflΔ deletion strain with different concentrations of 2,5-AM was measured, and it was found that 2,5-AM inhibited the growth of snflΔ deletion strain compared to wild-type strain (FIG. 9). This observed growth phenotype indicated that 2,5-AM undergoes glycolysis and becomes hydrolyzed into non-metabolized glycolytic intermediates.

2,5-AM is an analog of the sugar moiety fructose. Like glucose, fructose is also phosphorylated by hexokinase to form F6P. Phosphofructokinase converts F6P to fructose-1,6-bisphosphate (FBP), which is further metabolized into different downstream glycolytic intermediates. 2,5-AM can also be phosphorylated by hexokinase to form 2,5-AM-6-phosphate (2,5-AM6P). Furthermore, phosphofructokinase converts 2,5-AM6P to 2,5-AMBP. However, 2,5-AMBP cannot be further metabolized into downstream glycolytic intermediates.

Since 2,5-AM is a fructose analog, fructose could also extend the lifespan of yeast. Mannitol and maltose can also enter glycolysis and be metabolized into downstream glycolytic intermediates. To clarify whether other sugars also influence the CLS, the effects of fructose, mannitol and maltose on yeast aging were examined. Sorbitol, a non-metabolized sugar, was also tested as it is known to increase the CLS of yeast cells at high very high concentrations (18% equivalent to 1M) by increasing the osmolarity of the culture medium.

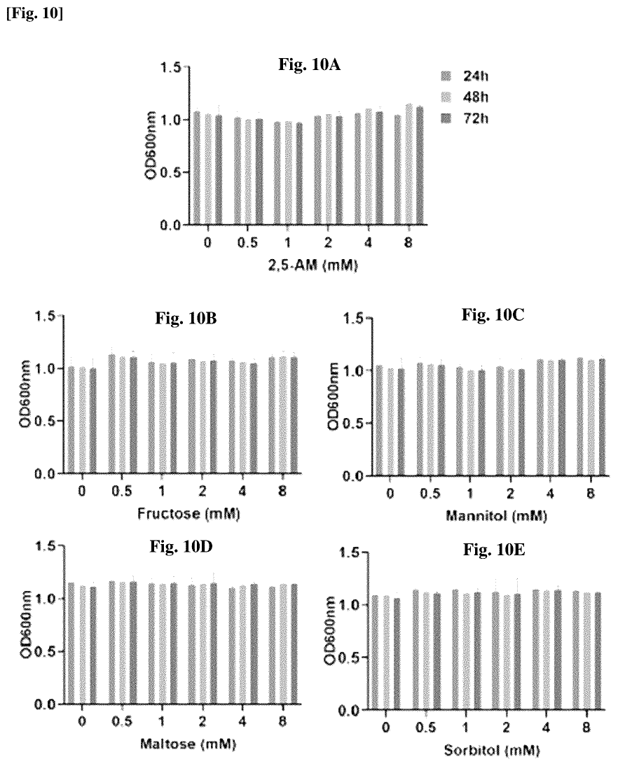

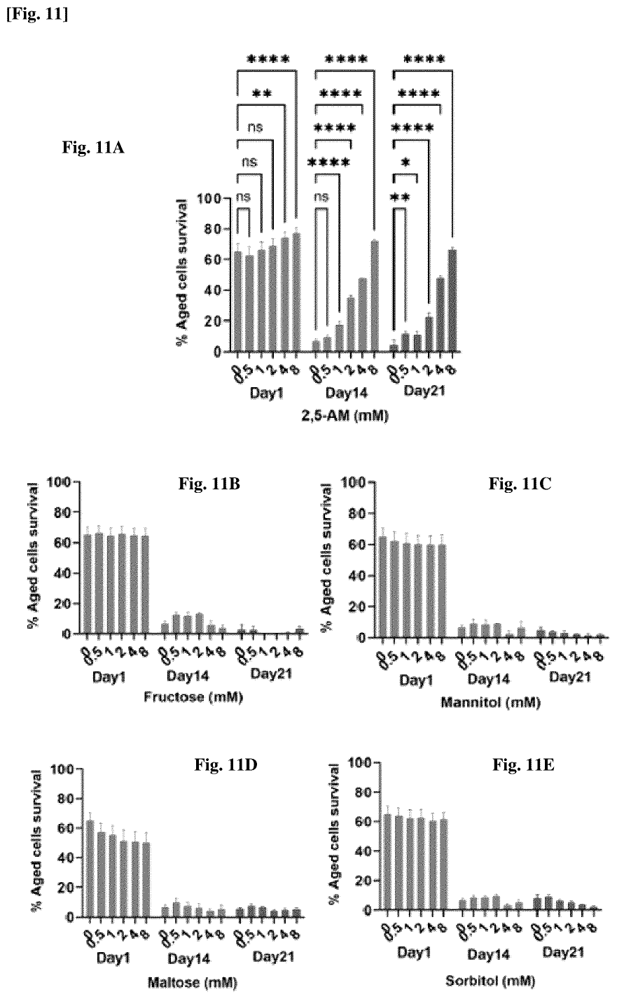

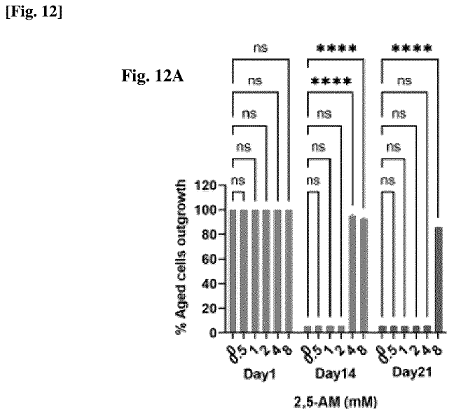

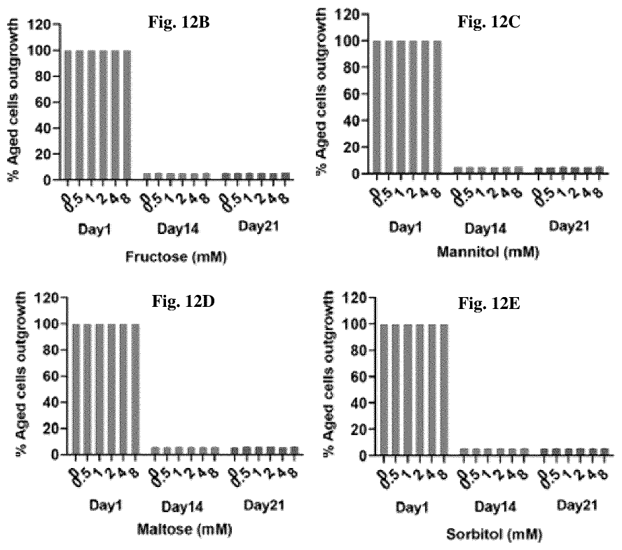

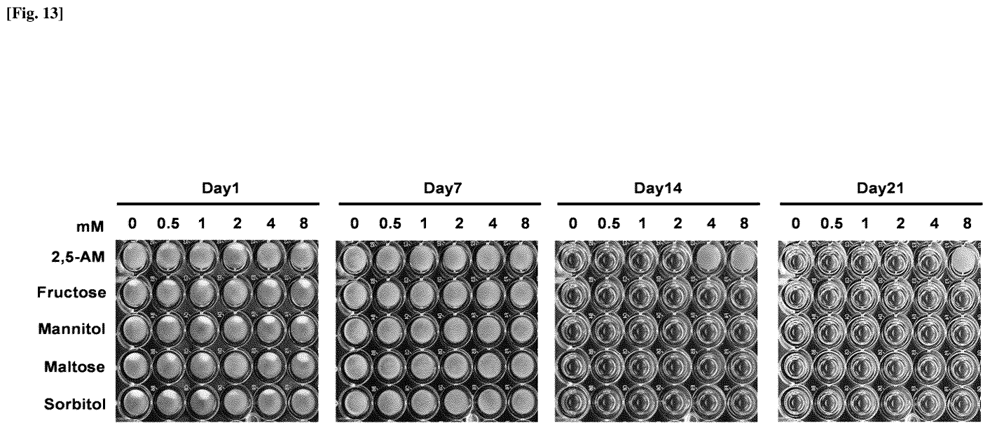

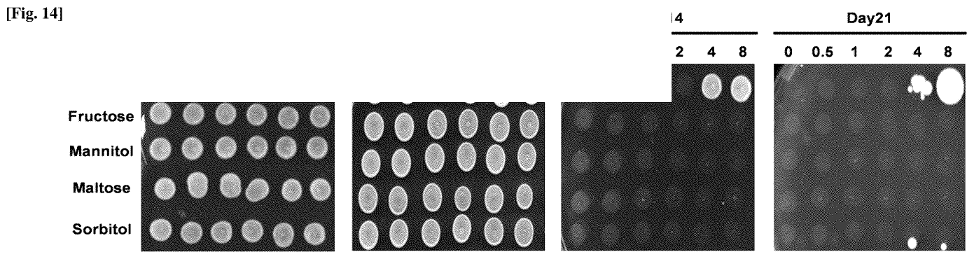

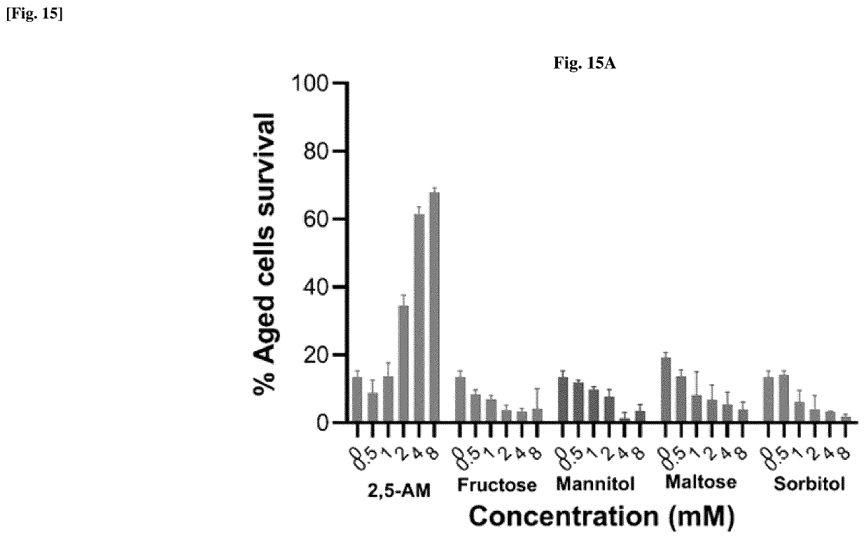

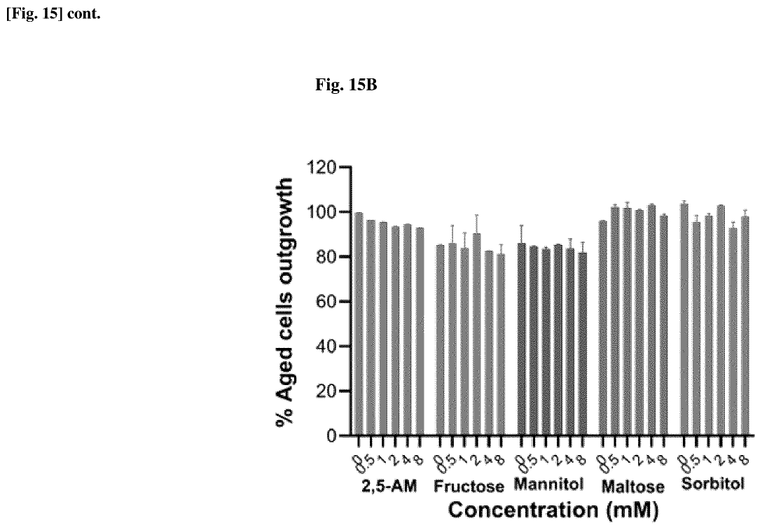

Firstly, the effect of fructose, mannitol, maltose, and sorbitol on cell growth was tested. Yeast cells were incubated with various concentrations of fructose, mannitol, maltose, and sorbitol similar to 2,5-AM in the SD medium. Like 2,5-AM, fructose, mannitol, maltose, and sorbitol incubated cells reached growth saturation after 24 hours (FIGS. 10A to E). Next, the survival of chronologically aged cells on day 1, day 7, day 14, and day 21 was measured. Unlike 2,5-AM, fructose, mannitol, maltose, and sorbitol supplementation did not extend the lifespan of the yeast (FIGS. 11 to 15). Remarkably, 2,5-AM extended the lifespan even at the late stage of chronological aging. Aged cells supplemented with 2,5-AM (8 mM) survived (approximately 65%) on Day 21. However, the survival of aged cells without 2,5-AM supplementation was less than 10%.

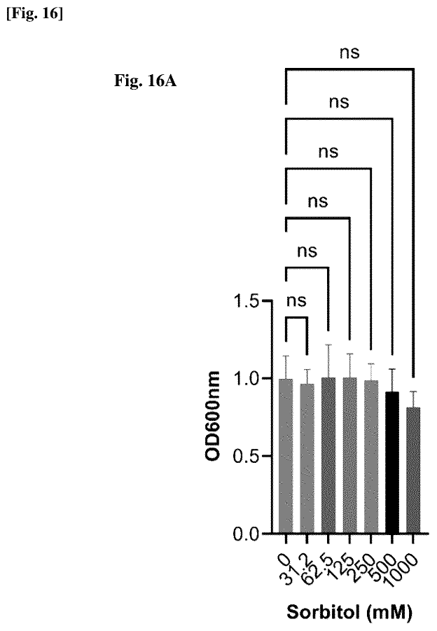

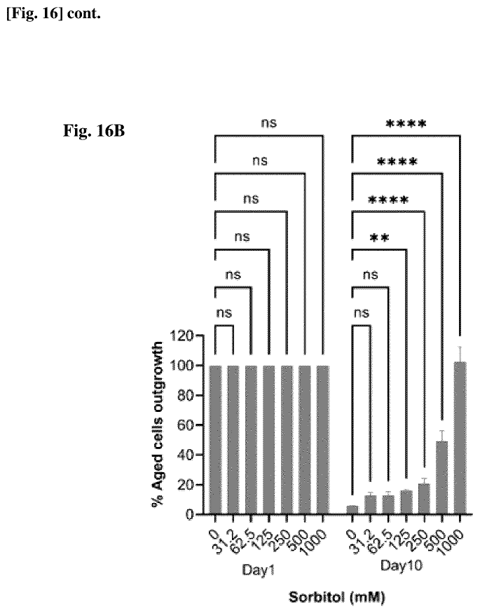

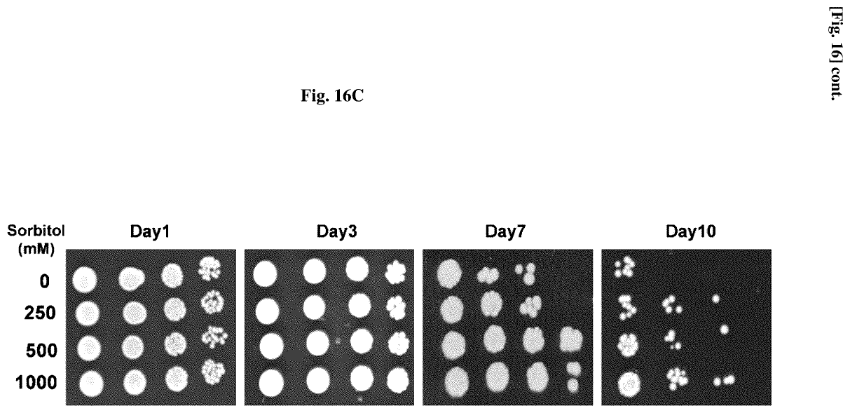

It was also found that surprisingly, sorbitol at low concentrations was unable to increase the CLS (FIGS. 11 to 15). It was hypothesized that higher sorbitol concentrations might be required to increase the CLS. As such, a range of higher concentrations including 1 M sorbitol, the concentration previously known to affect the CLS (18% equivalent to 1 M) was tested. Consistent with previous findings, it was found that very high sorbitol concentrations (between 0.5 and 1 M) increased the CLS of yeast (FIGS. 16A to C), apparently by increasing the osmolarity of the medium.

Interestingly, 2,5-AM increased CLS at very low concentrations (2 mM), suggesting its mechanism of anti-aging activity is distinct from the osmotic one displayed, for example, by sorbitol. These results suggested that the anti-aging activity of 2,5-AM is specific for this sugar compound and not observed for several other, similarly structured chemicals.

Target of Rapamycin Complex 1 (TORC1) and AMPK are the glucose-sensing complexes involved in the aging process. However, CLS extension by TORC1 inhibition is reduced when AMPK is inhibited. Glucose and glycolytic intermediates regulate the activity of TORC1 and AMPK. 2,5-AMBP has been shown to inhibit the activity of aldolase that catalyses the conversion of FBP into glyceraldehyde-3-phosphate (G3P) and dihydroxyacetone phosphate (DHAP). DHAP is interconverted into G3P by triosephosphate isomerase. Recent evidence suggests that DHAP is the important glucose signal molecule that activates TORC1.

Glycolytic flux is compromised when the syntheses of G3P and DHAP are affected, leading to less ATP production. Interestingly, AMPK is activated when the energy status of the cells is compromised and inhibits the cell growth by inhibiting TORC1. Thus, the antagonistic relationship of AMPK and TORC1 is an efficient metabolic adaptation to ensure cellular homeostasis for healthy cells. However, it was observed that 2,5-AM did not affect cell growth (FIGS. 4 to 8 and 10 to 14). Therefore, the possibility of 2,5-AM anti-aging activity via TORC1/AMPK or independently of TORC1/AMPK could not be excluded.

Unlike the inhibitory effect of 2,5-AMBP on aldolase, 2,5-AMBP is reported to activate the pyruvate kinase. Pyruvate kinase catalyses the conversion of phosphoenolpyruvate to pyruvate, one of the sources for generating nicotinamide adenine dinucleotide (NAD+). NAD+ is an important metabolite that regulates several cellular processes and functions critical to maintain healthy cells and extend the lifespan. Pyruvate is also a major substrate for mitochondrial reactions to generate various building block metabolites to synthesize vital elements essentially for cell survival and healthy aging, including amino acids, nucleic acids, and ATP. Indeed, the decline in mitochondrial activity is associated with aging and age-related diseases.

### Example 3: 2,5-Anhydro-D-mannitol Extended the Lifespan of Human Cells

The effect of 2,5-AM on the CLS of human cells were validated by three different methods.

### PI Method

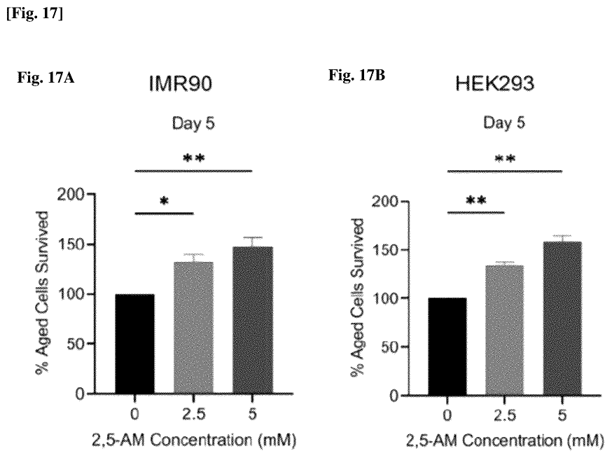

The anti-aging effect of 2,5-AM in various human cell lines was tested using the PI method. Firstly, the human lung primary fibroblast cells, IMR90, was tested. The primary fibroblast cells are widely used in aging research as they have a finite lifespan, and they better mimic the organism's aging process compared to immortal cell lines. 2,5-AM treatment significantly increased the cellular lifespan of IMR90 cells, resulting in a lower percentage of cell death. Both the 2.5 mM and the 5 mM treatment groups showed 32.33% and 48.08% more live cells compared to the untreated control, respectively (FIG. 17A).

It was also found that 2,5-AM increased cellular lifespan in the HEK293 cells, compared to the untreated group (FIG. 17B). The 2.5 mM and 5 mM treatment experiment showed 34.39% and 58.7% more live cells, respectively (FIG. 17A and 17B).

This showed that 2,5-AM is effective in increasing CLS in a variety of human cell lines.

### Crystal Violet Assay

In this approach, an outgrowth assay was used to determine the number of live cells in the 6-well experiment plate using crystal violet staining. Crystal violet is a triarylmethane dye that binds to ribose molecules such as DNA. The crystal violet-based outgrowth method relies on the detachment of adherent cells from the culture plate during cell death. Hence, dead cells would be washed away with the D10 medium and only live cells would remain attached to the culture plate. These live cells were then stained with crystal violet dye and assessed qualitatively.

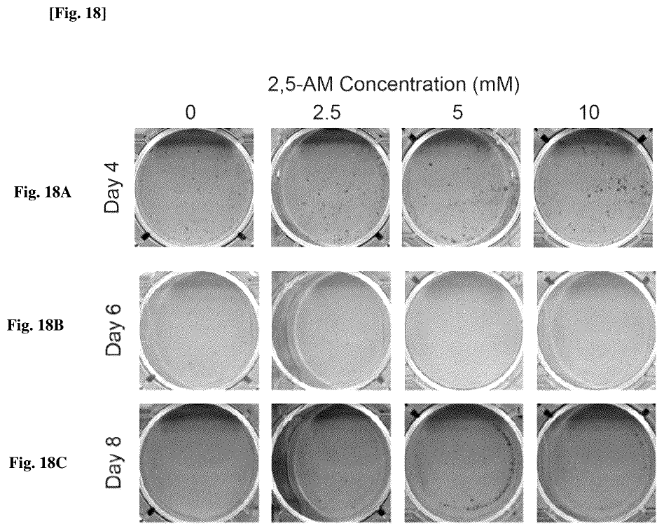

The cell viability of 2,5-AM treated cells was examined at different concentrations using the crystal violet-based CLS assay. It was observed that after seven days of outgrowth, 2,5-AM was able to increase cellular lifespan in HEK293 cell line (FIG. 18A). Similar results could be observed on the Day 8 culture (FIG. 18C) but not on the Day 6 culture (FIG. 18B). This may be due to the variation in washing steps during the crystal violet staining. Nonetheless, these results further proved that 2,5-AM can increase cellular lifespan in the human cell line, confirming its conserved anti-aging properties over vast eukaryote taxonomic ranges.

### PrestoBlue™ Outgrowth Assay

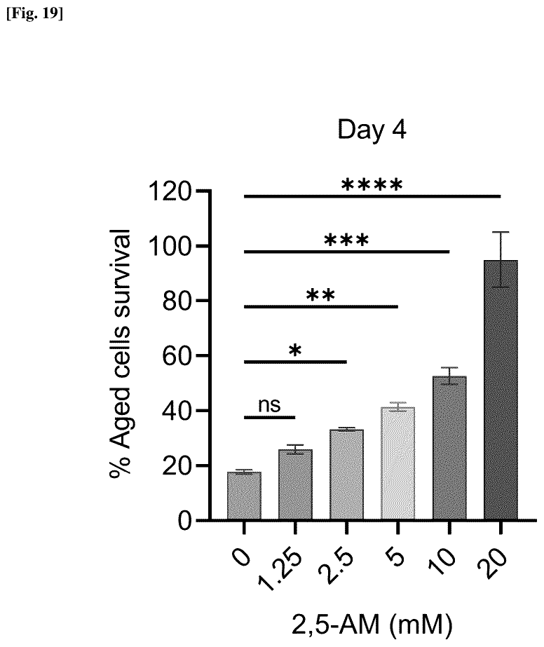

The effect of 2,5-AM supplementation on the CLS of human embryonic kidney 293 (HEK293) cells was tested using an outgrowth method. Trends of lifespan extension by 2,5-AM in human HEK293 cells (FIG. 19) was observed, further showing that 2,5-AM may be used to extend CLS in human cells.

PrestoBlue (PB) is a resazurin-based reagent used to detect cell viability and traces of cytotoxicity in the medium. It produces results that can be detected both colorimetrically and fluorometrically. It is permeable and non-toxic to the cells, hence, enabling the measurement of cell viability over a period of time without cell lysis. The resazurin component is reduced to form resorufin by live cells, resulting in a visible change from blue colour to red colour in the reagent, at the same time developing fluorescence, which can be easily detected by a microplate reader. The PB reagent is highly sensitive; it detects even a small number of cells (a couple of hundreds). It allows conveniently fast cell detection and data collection. The PB assay was easy to perform as the reagent is a commercially available as ready-to-use solution.

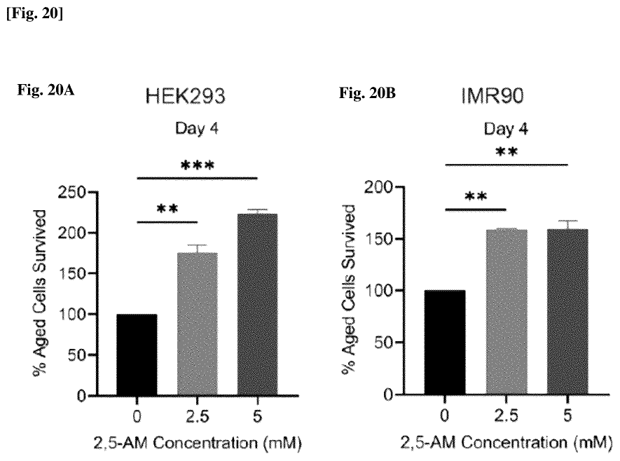

In the outgrowth assay using human cell lines, 2,5-AM treatment significantly increased the cellular lifespan, which resulted in more live cells that were able to proliferate in the assay plate. For the HEK293 cell line, the 2.5 mM treatment group had 76.31% more viable cells compared to the untreated group (FIG. 20A). Moreover, the 5 mM treatment group had even 123.7% more surviving cells relative to the untreated group (FIG. 20B).

The anti-aging effect of 2,5-AM in the human lung primary fibroblast cells IMR90, was also tested. An anti-aging effect similar to that for HEK293 was also observed for the IMR90 cells. The 2.5 mM treatment group had 59.05% more surviving cells and the 5 mM treatment group had 59.89% more viable cells compared to the untreated group (FIG. 20B).

These results further proved that 2,5-AM can extend cellular lifespan in the human cell line.

Thus, it was confirmed that 2,5-AM is a novel anti-aging compound that can increase cellular lifespan across different species from yeast to human cells. Based on the findings, 2,5-AM may be used individually or in combination with other anti-aging interventions.

### Example 4: High-Throughput Chemical Screening of Plicamycin, Copanlisib Dihydrochloride, and Nedaplatin using the PI Method in Human Cells

The PI method was used to identify additional novel anti-aging compounds that prolong CLS. A total of 162 compounds were screened, including some FDA-approved drugs and natural products. The log₁₀ values of the PI intensity for the various control samples ranged from 4.22 to 4.53. Hence, compounds with clearly lower log PI intensity were viewed to extend CLS. In addition to the known anti-aging compounds rapamycin and 2,5-AM that were added to the screen as positive controls, three other compounds (plicamycin, copanlisib dihydrochloride, and nedaplatin) exhibited a low log₁₀ PI intensity, indicating new candidate anti-aging compounds.

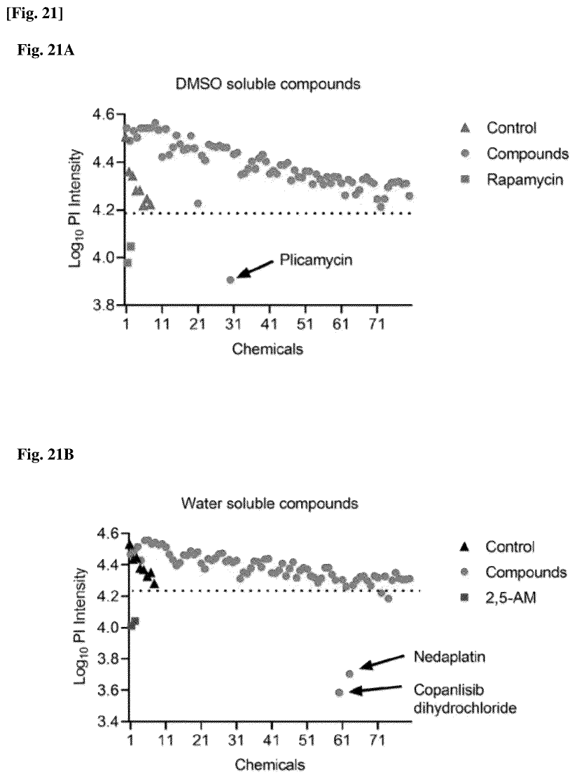

Among the DMSO-soluble compounds, plicamycin was detected to increase CLS, with log₁₀ PI intensity of 3.91 on Day 6 (FIG. 21A). From the set of water-soluble compounds tested, copanlisib dihydrochloride and nedaplatin were also demonstrated to have anti-aging properties with log₁₀ PI intensities of 3.59 and 3.70, respectively (FIG. 21B).

Plicamycin, copanlisib dihydrochloride and nedaplatin are potential anti-cancer agents. However, the potential of these compounds in extending CLS has not yet been reported, and the known anti-cancer mechanism of action of these drugs might not be centrally relevant to cellular lifespan extension.

Plicamycin is identified to bind to the GC-rich sequences in DNA, prevents transcription factors from complexing with promoters, and inhibits RNA synthesis. Nedaplatin, a derivative of cisplatin, binds to DNA and forms a cross-link, which inhibits DNA synthesis and replication.

Copanlisib dihydrochloride is a phosphoinositide 3-kinase (PI3K) inhibitor. PI3K activation enables the tumour to evade immune detection. Notably, PI3K positively regulates the TORC1 pathway, which may result in CLS extension. TORC1 is a eukaryotic protein complex that is conserved from yeast to humans and couples the presence of nutrients with DNA replication, transcription, and translation. TORC1 promotes cellular anabolic processes such as the synthesis of nucleotides and proteins, and it inhibits catabolic processes, including oxidative phosphorylation and autophagy.

TORC1 positively regulates the aging process. The clinically approved drug rapamycin inhibits TORC1 and extends the lifespan and healthspan of murines. Rapamycin (sirolimus) and its analogs, everolimus (afinitor) and temsirolimus (torisel), are currently applied in the treatment for a few chronic diseases, including cancer. Rapamycin is in clinical trials for its use as an anti-aging therapeutic. Mechanistically, it is hypothesized that plicamycin, copanlisib dihydrochloride, and nedaplatin may exert their anti-aging properties through modulation of TORC1 or direct translation.

### Example 5: Extension of Cellular Lifespan with FDA-Approved Drugs, Hemin, 5-fluorouracil, Atorvastatin, and Methotrexate

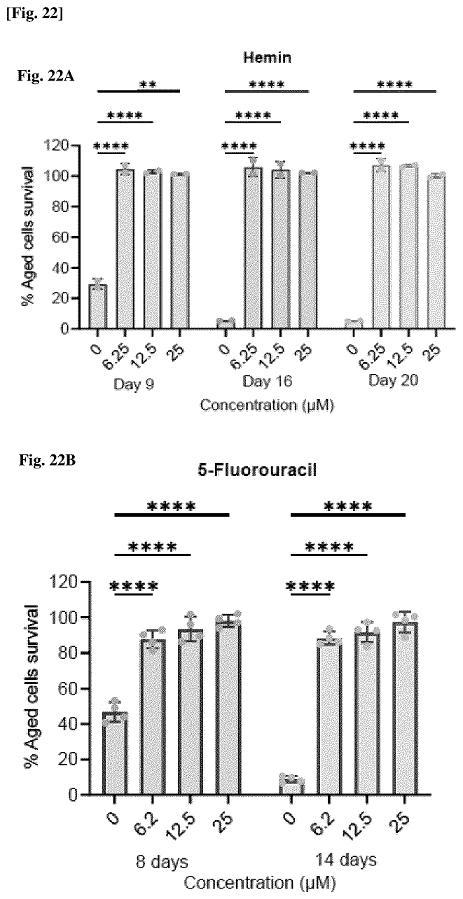

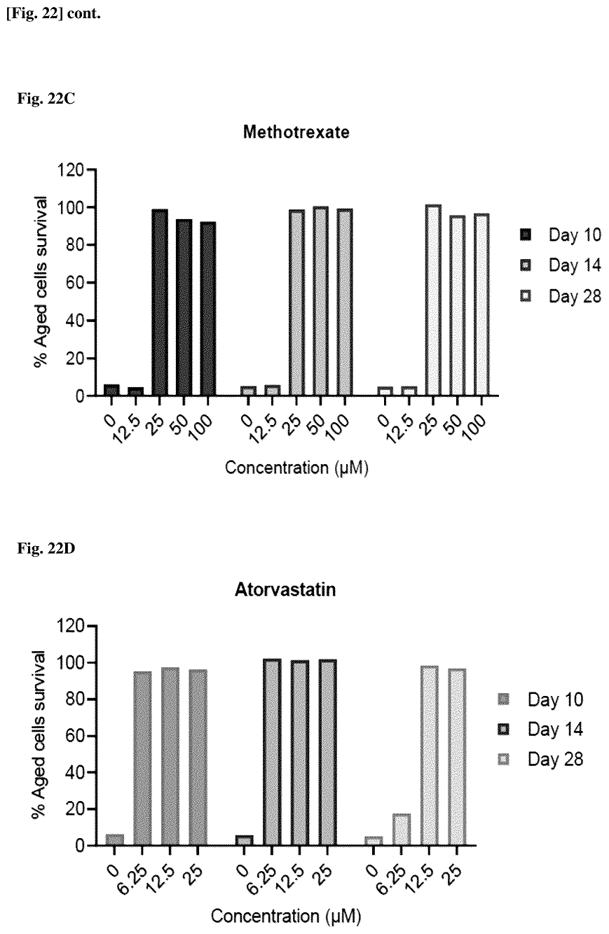

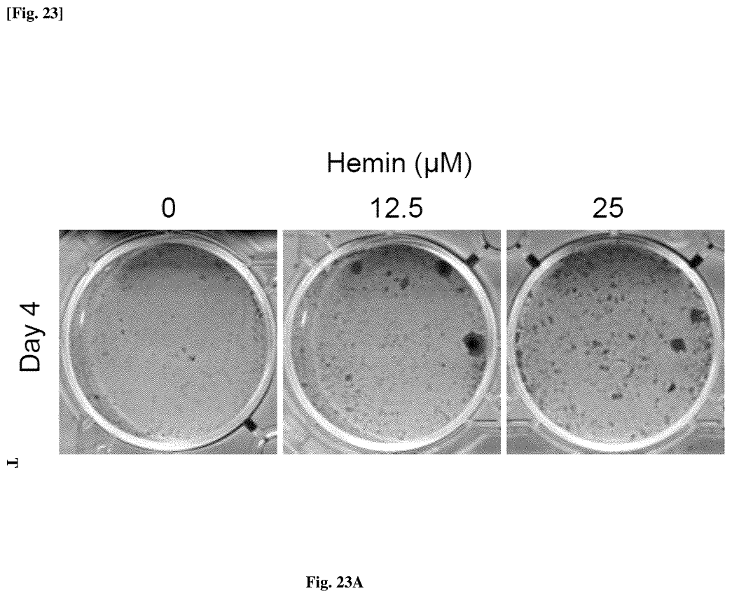

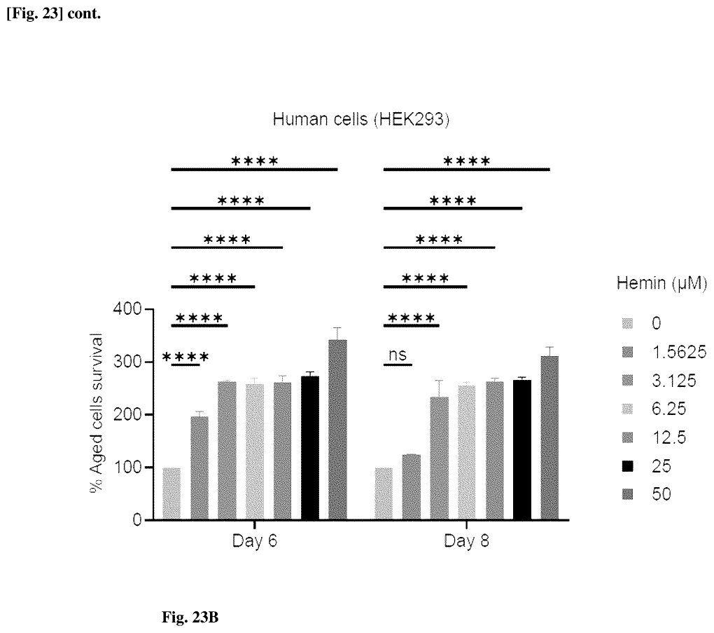

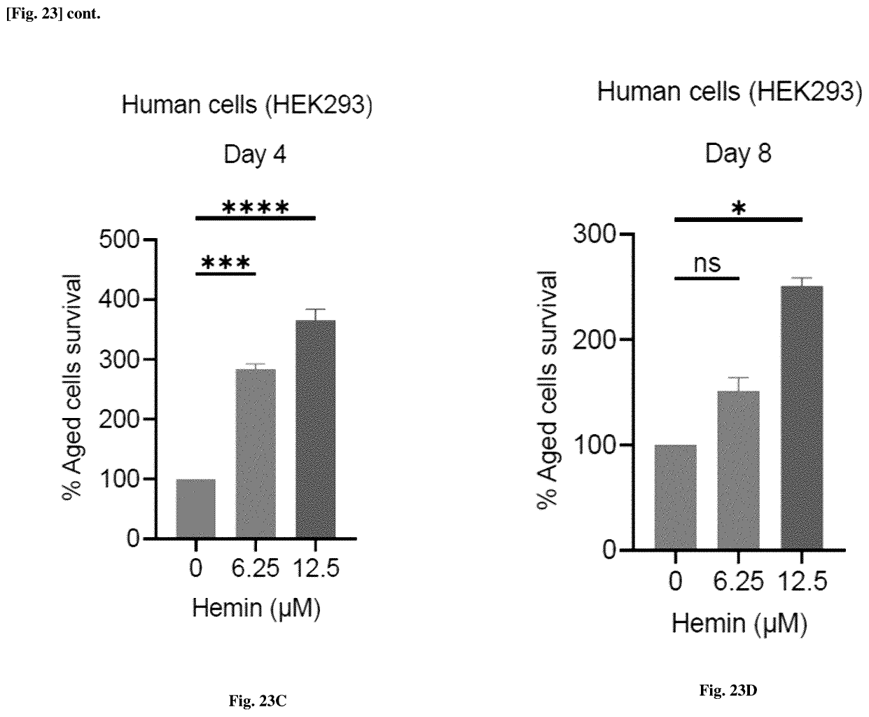

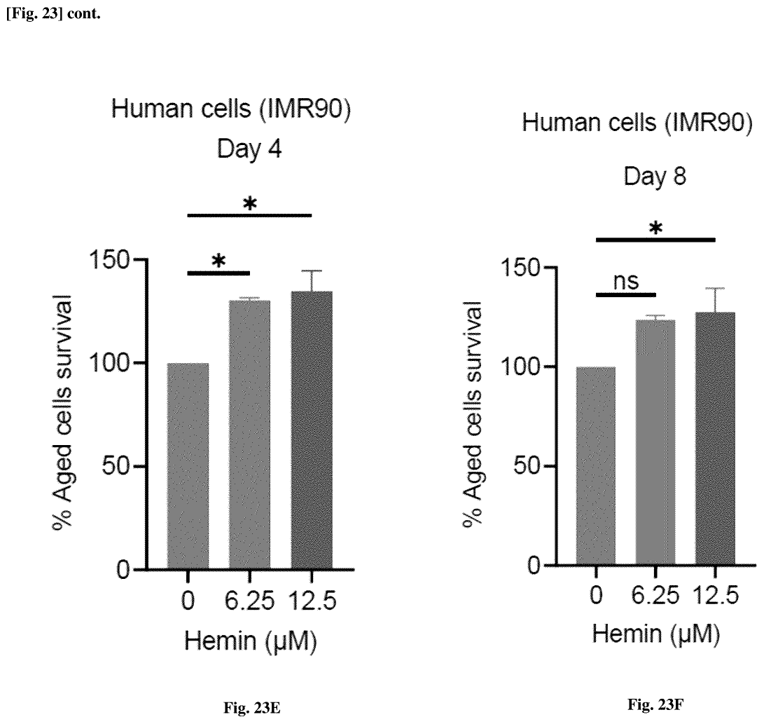

The anti-aging activity of several drugs approved by the US Food and Drug Administration (FDA) (i.e., hemin, 5-fluorouracil, atorvastatin, and methotrexate) was tested in the aging yeast model of Saccharomyces cerevisiae. Cells that were supplemented with various concentrations of these drugs survived longer as compared to those without treatment (FIGS. 22A to D). The longevity effect of hemin in human cell lines HEK293 and IMR90 was also tested using various aging assays. Similar cellular lifespan extension patterns were found with hemin in human cells (FIGS. 23A to F) using the PrestoBlue™ outgrowth assay.

### INDUSTRIAL APPLICABILITY

The compound selected from the group consisting of a compound having the formula (I), plicamycin, copanlisib dihydrochloride, nedaplatin, hemin, 5-fluorouracil, atorvastatin, methotrexate, and any mixture thereof, as defined herein, may be used as a novel anti-aging compound to delay the aging process (via cellular lifespan extension and post-mitotic survival) of yeast, bacteria and animals. In animals such as mammals, the compound as defined above may be useful in alleviating any age-related ailment (e.g., diabetic complications, retinopathy, atherosclerosis, hypertension, obesity, cancer, benign prostate hyperplasia, Alzheimer and Parkinson diseases, age-related macular degeneration, osteoarthritis, osteoporosis, sarcopenia, and seborrheic keratosis), thereby extending the healthy lifespan of the animals with youthful characteristics. The compound as defined herein may be utilized individually or in combination to supplement current medical approaches (i.e., exercise and diet) to better manage aging and enhance an individual's lifestyle. The compound as defined herein may significantly improve the quality of life and reduce overall healthcare costs, especially in the elderly. The compound as defined herein may also be used as a positive control for extending CLS in developing new technologies to measure cellular lifespan.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1.-18. (canceled)

19. A method of extending the chronological lifespan of a cell, comprising the step of contacting a cell with a compound selected from the group consisting of a compound having the following formula (I), plicamycin, copanlisib dihydrochloride, nedaplatin, hemin, 5-fluorouracil, atorvastatin, methotrexate, and any mixture thereof: 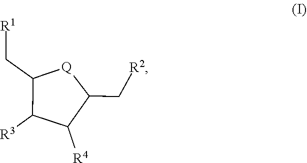
wherein in formula (I),
R¹, R², R³ and R⁴ is each independently -QH, ——NR⁵R⁶, or —OC(O)R⁵;
R⁵ and R⁶ is each independent-H or an optionally substituted alkyl; and
Q is O or S.

20. The method according to claim 19, wherein the cell is contacted with the compound selected from the group consisting of the compound of formula (I), plicamycin, copanlisib dihydrochloride, nedaplatin, hemin, 5-fluorouracil, atorvastatin, methotrexate, and any mixture thereof.

21. The method according to claim 19, wherein the compound of formula (I) has the following formula (Ia), (Ib) or (Ic):

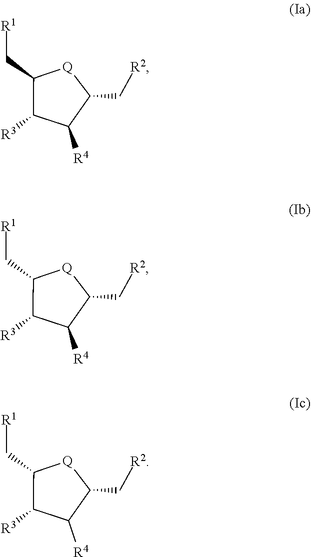

22. The method according to claim 19, wherein R¹, R², R³ and R⁴ of the compound of formula (I) is each independently selected from the group consisting of —OH, —O—C(O)—CH₃ and —NH₂.

23. The method according to claim 19, wherein R¹, R², R³ and R⁴ of the compound of formula (I) are all —OH.

24. The method according to claim 19, wherein R¹, R², R³ and R⁴ of the compound of formula (I) are all —OC(O)R⁵.

25. The method according to claim 19, wherein R¹ of the compound of formula (I) is NH₂ and R², R³ and R⁴ of the compound of formula (I) are all —OH.

26. The method according to claim 19, wherein Q of the compound of formula (I) is O.

27. The method according to claim 19, wherein the compound of formula (I) is selected from the group consisting of 2,5-anhydro-D-mannitol, 2,5-anhydro-D-mannitol tetraacetate, 2,5-ahydro-D-glucitol, and 1-amino-2,5-anhydro-D-glucitol.

28. The method according to claim 19, wherein the compound of formula (I) is selected from the group consisting of:

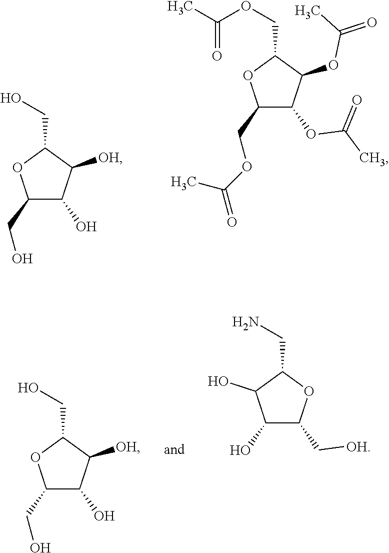 [SMILES: OC[C@H]1O[C@H](CO)[C@@H](O)[C@@H]1O ; CC(=O)OC[C@H]1O[C@H](COC(C)=O)[C@@H](OC(C)=O)[C@@H]1OC(C)=O ; OC[C@@H]1O[C@H](CO)[C@@H](O)[C@@H]1O ; NC[C@@H]1O[C@H](CO)[C@H](O)C1O]

29. The method according to claim 19, wherein the compound selected from the group consisting of the compound of formula (I), plicamycin, copanlisib dihydrochloride, nedaplatin, hemin, 5-fluorouracil, atorvastatin, methotrexate, and any mixture thereof, is contacted with the cell at various concentrations.

30. The method according to claim 19, wherein the compound selected from the group consisting of the compound of formula (I), plicamycin, copanlisib dihydrochloride, nedaplatin, hemin, 5-fluorouracil, atorvastatin, methotrexate, and any mixture thereof, is contacted with the cell for a various duration.

31. The method according to claim 19, wherein the cell is a fungal cell, bacterial cell or animal cell.

32. The method according to claim 19, wherein the extension of the chronological lifespan of the cell results in treatment of an aging-associated condition in a subject in need thereof.

33. The method of claim 32, wherein the aging-associated condition is selected from the group consisting of diabetic complications, retinopathy, atherosclerosis, hypertension, obesity, cancer, benign prostate hyperplasia, Alzheimer and Parkinson diseases, age-related macular degeneration, osteoarthritis, osteoporosis, sarcopenia and seborrheic keratosis.
